(19) United States
(12) Patent Application Publication
(10) Pub. No.: US 2006/0269971 A1
(43) Pub. Date: Nov. 30, 2006

(54) Methods for detecting prostate cancer

(75) Inventor: Eleftherios P. Diamandis, Toronto (CA)

Correspondence Address: MERCHANT & GOULD PC, P.O. BOX 2903, MINNEAPOLIS, MN, 55402-0903, US

(73) Assignee: Mount Sinai Hospital, Toronto (CA)

(21) Appl. No.: 10/529,088

(22) PCT Filed: Sep. 26, 2003
(86) PCT No.: PCT/CA03/01479

§ 371 Date: Aug. 4, 2006

(51) Int. Cl. G01N 33/574; G06Q 50/00; G06F 19/00

(52) U.S. Cl. 435/7.23; 705/2; 702/19

(57) ABSTRACT

The invention relates to the application of kallikrein 11, free PSA, and total PSA in the detection of prostate cancer. These markers may be used for the diagnosis, monitoring, staging, progression, prevention, treatment, and prognosis of prostate cancer, and as indicators before surgery or after relapse. A particular aspect of the invention provides a method for distinguishing prostate cancer from benign prostatic hyperplasia (BPH).

### FIELD OF THE INVENTION

The invention relates to kallikrein 11, prostate-specific antigen, free prostate-specific antigen and their use in detecting prostate cancer.

### BACKGROUND OF THE INVENTION

Prostate cancer (CaP) is the most frequently diagnosed cancer in men in North America and its mortality rate is second only to lung cancer. Therefore, early diagnosis and monitoring of prostate cancer is an important priority. Prostate-specific antigen (PSA) is widely used as the most reliable tumor marker established so far^{1,2}. However, non-malignant prostatic diseases, especially benign prostatic hyperplasia (BPH) and acute prostatitis also cause serum PSA elevation, thus complicating the diagnosis of prostate cancer by PSA measurements alone. Analysis of the molecular forms of PSA improves specificity for prostate cancer^{3,4}. The determination of free PSA and its ratio to total PSA is now clinically established and is used to reduce the number of unnecessary prostate biopsies. Despite numerous efforts to further reduce unnecessary biopsies, false negative and false positive results still occur with high frequency.

The human kallikrein 11 gene (KLK11) was originally isolated from human hippocampus as trypsin-like serine protease (TLSP)⁶. Two alternative splice variants of this gene, also known as hippostasin have also been identified⁷⁻⁹. With the official nomenclature, TLSP/hippostasin is now known as human kallikrein 11 (hK11). This protein is encoded by the KLK11 gene that belongs to the human kallikrein family along with PSA (hK3) and other kallikreins¹⁰. hK11 protein has been found to be highly expressed in the prostate^{8,11}.

The citation of any reference herein is not an admission that such reference is available as prior art to the instant invention.

### SUMMARY OF THE INVENTION

The invention relates to the application of kallikrein 11, free PSA, and total PSA in the detection of prostate cancer. These markers may be used for the diagnosis, monitoring, staging, progression, prevention, treatment, and prognosis of prostate cancer, and as indicators before surgery or after relapse.

Kallikrein 11, and PSA, and agents that bind to kallikrein 11 and PSA, may be used to detect prostate cancer and they can be used in the diagnostic evaluation of prostate cancer, and the identification of subjects with a predisposition to such disorders. Methods for detecting kallikrein 11 and PSA can be used to monitor prostate cancer.

The presence of kallikrein 11 and PSA in a sample can be assessed, for example, by detecting the presence in the sample of (a) kallikrein 11 and PSA or fragments thereof; or (b) metabolites which are produced directly or indirectly by a kallikrein 11 or PSA.

In an aspect, the invention provides a method for detecting prostate cancer in a subject comprising measuring kallikrein 11 and prostate specific antigen (PSA) in a sample from the subject.

In an embodiment, the invention provides a method for detecting kallikrein 11 and PSA comprising (a) obtaining a sample from a patient; (b) detecting or identifying in the sample kallikrein 11 and PSA; and (c) comparing the detected amounts with amounts detected for a standard.

In an aspect of the invention, a method for screening a subject for prostate cancer is provided comprising (a) obtaining a biological sample from a subject; (b) detecting the amounts of kallikrein 11, free PSA, and total PSA in said sample; and (c) comparing said amounts of kallikrein 11, free PSA, and total PSA detected to predetermined standards, where detection of different levels of kallikrein 11, free PSA, and total PSA compared with that of a standard indicates disease.

The terms “detect”, “detecting” and “detection” include assaying or otherwise establishing the presence or absence of kallikrein 11, a combination of kallikrein 11 and PSA, subunits thereof, or combinations of reagent bound targets, and the like, or assaying for, imaging, ascertaining, establishing, or otherwise determining one or more factual characteristics of prostate cancer, metastasis, stage, or similar conditions. The term encompasses diagnostic, prognostic, and monitoring applications.

According to a method involving kallikrein 11 and PSA, the levels in a sample from a patient are compared with the normal levels of kallikrein 11 and PSA in samples of the same type obtained from controls (e.g. samples from individuals not afflicted with disease). Significantly altered levels in the sample of kallikrein 11 and PSA relative to the normal levels in a control is indicative of disease. Thus, a method is provided of assessing whether a patient is afflicted with or has a pre-disposition to prostate cancer, comprising comparing (a) levels of kallikrein 11 and PSA in a sample from the patient; and (b) normal levels of kallikrein 11 and PSA in samples of the same type obtained from control patients not afflicted with prostate cancer, wherein significantly altered levels of kallikrein 11 and PSA relative to the corresponding normal levels of kallikrein 11 and PSA, is an indication that the patient is afflicted with or has a pre-disposition to prostate cancer.

A significant difference between the levels of kallikrein 11 and PSA in the patient and the normal levels (e.g. lower levels in the patient) is an indication that the patient is afflicted with or has a predisposition to prostate cancer.

The present invention also relates to a method for diagnosing and monitoring prostate cancer in a subject comprising detecting or measuring kallikrein 11 and PSA in a sample from the subject using a reagent that detects kallikrein 11 and PSA, in particular antibodies specifically reactive with kallikrein 11 and PSA or a part thereof. In an embodiment, the sample is serum.

In an embodiment, the invention relates to a method for diagnosing and monitoring prostate cancer in a subject by quantitating kallikrein 11 and PSA in a biological sample from the subject comprising (a) reacting the biological sample with antibodies specific for kallikrein 11 and PSA which is directly or indirectly labelled with a detectable substance; and (b) detecting the detectable substance.

In another aspect the invention provides a method for using antibodies to detect expression of a kallikrein 11 and PSA in a sample, the method comprising: (a) combining antibodies specific for kallikrein 11 and PSA with a sample under conditions which allow the formation of antibody:protein complexes; and (b) detecting complex formation, wherein complex formation indicates expression of kallikrein 11 and PSA in the sample. Expression may be compared with standards and is diagnostic of prostate cancer.

Embodiments of the methods of the invention involve (a) reacting a biological sample from a subject with antibodies specific for kallikrein 11 and PSA which are directly or indirectly labelled with an enzyme; (b) adding a substrate for the enzyme wherein the substrate is selected so that the substrate, or a reaction product of the enzyme and substrate forms fluorescent complexes; (c) quantitating kallikrein 11 and PSA in the sample by measuring fluorescence of the fluorescent complexes; and (d) comparing the quantitated levels to levels obtained for other samples from the subject patient, or control subjects. In an embodiment the quantitated levels are compared to levels quantitated for control subjects without prostate cancer wherein lower levels of kallikrein 11 and PSA compared with the control subjects is indicative of prostate cancer.

A particular embodiment of the invention comprises the following steps
(a) incubating a biological sample with first antibodies specific for kallikrein 11 and PSA which are directly or indirectly labeled with detectable substances, and second antibodies specific for kallikrein 11 and PSA which are immobilized;
(b) detecting the detectable substances thereby quantitating kallikrein 11 and PSA in the biological sample; and
(c) comparing the quantitated kallikrein 11 and PSA with levels for a predetermined standard.

The standard may correspond to levels quantitated for samples from control subjects without prostate cancer, with a different disease stage, or from other samples of the subject. Lower levels of kallikrein 11 and PSA as compared to the standard is indicative of prostate cancer.

The invention also contemplates the methods described herein using multiple markers for prostate cancer. Therefore, the invention contemplates a method for anaylzing a biological sample for the presence of kallikrein 11 and PSA and other markers that are specific indicators of prostate cancer. The methods described herein may be modified by including reagents to detect the markers, or nucleic acids for the markers.

The invention further relates to a method of assessing the efficacy of a therapy for inhibiting prostate cancer in a patient A method of the invention comprises comparing: (a) levels of kallikrein 11 and PSA in a sample from the patient obtained from the patient prior to providing at least a portion of the therapy to the patient; and (b) levels of kallikrein 11 and PSA in a second sample obtained from the patient following therapy.

A significant difference between the levels of kallikrein 11 and PSA in the second sample relative to the first sample (e.g. higher levels of kallikrein 11 and PSA) is an indication that the therapy is efficacious for inhibiting prostate cancer.

The “therapy” may be any therapy for treating prostate cancer including but not limited to therapeutics, radiation, immunotherapy, gene therapy, and surgical removal of tissue. Therefore, the method can be used to evaluate a patient before, during, and after therapy.

In an aspect, the invention provides a method for monitoring the progression of prostate cancer in a patient the method comprising:
(a) detecting kallikrein 11 and PSA in a sample from the patient at a first time point;
(b) repeating step (a) at a subsequent point in time; and
(c) comparing the levels detected in (a) and (b), and therefrom monitoring the progression of the prostate cancer.

The invention also provides a method for assessing the potential efficacy of a test agent for inhibiting prostate cancer, and a method of selecting an agent for inhibiting prostate cancer.

The invention also contemplates a method of assessing the potential of a test compound to contribute to prostate cancer comprising:
(a) maintaining separate aliquots of prostate cancer diseased cells in the presence and absence of the test compound; and
(b) comparing the levels of kallikrein 11 and PSA in each of the aliquots.

A significant difference between the levels of kallikrein 11 and PSA in the aliquot maintained in the presence of (or exposed to) the test compound relative to the aliquot maintained in the absence of the test compound, indicates that the test compound potentially contributes to prostate cancer.

In the methods of the invention the amount of kallikrein 11 and the amount of PSA may be mathematically combined. In an embodiment of the invention, a method is provided for detecting or identifying prostate cancer in a subject comprising:
(a) determining the amount of kallikrein 11 in a sample from the subject;
(b) determining the amount of PSA in the sample;
(c) mathematically combining the results of step (a) and step (b); and
(d) relating the combination to the presence of prostate cancer.

In aspects of the invention, the combination is a ratio of kallikrein 11 to total PSA, or the inverse thereof. The combination is preferably compared to a mathematical combination for a predetermined standard.

In particular, the invention provides a method for screening or identifying prostate cancer by determining the ratio between kallikrein 11:total PSA in a sample from a subject, preferably a serum sample.

A method of the invention, in particular a method for detecting or identifying prostate cancer, may further comprise determining the % free PSA and relating the combination and % free PSA to the presence of prostate cancer.

In an aspect the invention provides a method for distinguishing prostate cancer from benign prostatic hyperplasia (BPH) in a subject comprising determining the amount of kallikrein 11 contained in a sample from the subject, and relating the amount to the presence of prostate cancer or BPH in the subject. The amount of kallikrein 11 in the sample may be compared to an amount determined for a standard. A standard may be an amount of kallikrein 11 associated with prostate cancer, and a higher amount of kallikrein 11 in the sample compared to the standard may be indicative of BPH. A standard may be an amount of kallikrein 11 associated with BPH, and a lower amount of kallikrein 11 in the sample compared to the standard may be indicative of prostate cancer.

In a further aspect, the invention provides a method for distinguishing prostate cancer from benign prostatic hyperplasia (BPH) in a subject comprising:
(a) determining the amount of kallikrein 11 contained in a sample from the subject;
(b) determining the amount of total PSA contained in the sample;
(c) mathematically combining the results of (a) and (b);
(d) relating the combination to the presence of BPH or prostate cancer.

The combination may be a ratio of kallikrein 11 to total PSA, or the inverse thereof.

Therefore, the invention provides a method for differentiation between BPH or prostate cancer by determining the ratio between kallikrein 11:total PSA in a subject's serum.

The combination (e.g. ratio) may be compared to a combination for a standard associated with prostate cancer or BPH. A standard may be a combination (e.g. ratio) determined for prostate cancer, and a higher combination (e.g. ratio) determined for the sample compared to the standard may be indicative of BPH. A standard may be a combination (e.g. ratio) determined for BPH, and a lower combination determined for the sample compared to the standard may be indicative of prostate cancer.

The methods of the invention may further comprise determining the percentage of free PSA and correlating the percentage free PSA and the combination to the presence of prostate cancer or BPH in the subject.

In the methods of the invention kallikrein 11 and PSA may be measured using agents that bind kallikrein 11 and PSA, respectively. The binding agents may be directly or indirectly labeled with a detectable substance.

In particular, the invention provides a diagnostic method for determining the presence of BPH or prostate cancer in a subject comprising:
(a) contacting a sample from the subject with a first binding agent that binds to kallikrein 11 and a second binding agent that binds to PSA;
(b) determining the presence or amount of first complexes comprising the first agent and kallikrein 11 and second complexes comprising the second agent and PSA;
(c) mathematically combining the amount of the first and second complexes; and
(d) relating the combination to the presence of prostate cancer.

In particular methods of the invention, patients with BPH or prostate cancer can be identified in patients with total PSA between about 4 to 10 ng/ml.

In still other particular methods of the invention, patients with BPH or prostate cancer can be identified in patients with total PSA less than 4 ng/ml.

The invention also provides a method of improving the accuracy of a diagnosis of prostate cancer comprising the steps of: a) performing a method of the invention; and b) performing at least one of a test for free PSA and a digital rectal examination.

The invention also relates to kits for carrying out the methods of the invention. In an embodiment, the kit is for assessing whether a patient is afflicted with prostate cancer, and it comprises reagents for assessing kallikrein 11 and PSA.

In another aspect, the invention relates to a kit for assessing the suitability of each of a plurality of test compounds for inhibiting prostate cancer in a patient The kit comprises reagents for assessing kallikrein 11 and PSA and optionally a plurality of test agents or compounds.

The invention contemplates a kit for assessing the presence of prostate cancer cells, wherein the kit comprises antibodies specific for kallikrein 11 and PSA, and optionally antibodies specific for other markers associated with prostate cancer.

Additionally the invention provides a kit for assessing the potential of a test compound to contribute to prostate cancer. The kit comprises prostate cancer cells and reagents for assessing kallikrein 11 and PSA, and optionally other markers associated with prostate cancer.

The invention also provides a diagnostic composition comprising kallikrein 11 and PSA or agents that bind to kallikrein 11 and PSA or parts thereof. Agents can be labelled with detectable substances.

The invention also contemplates the methods, compositions, and kits described herein using additional markers associated with prostate cancer. The methods described herein may be modified by including reagents to detect the additional markers, or nucleic acids for the markers.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The above-mentioned and other features of this invention and the manner of obtaining them will become more apparent, and will be best understood, by reference to the following description, taken in conjunction with the accompanying drawings. These drawings depict only a typical embodiment of the invention and do not therefore limit its scope. They serve to add specificity and detail, in which:

FIG. 1 shows the distribution of hK11/total PSA ratio in BPH and CaP patients. At 90% sensitivity (hK11/total PSA ratio of 0.06) the specificity is 48.5%. P value was determined by the Mann-Whitney U test.

FIG. 2 shows the distribution of hK11/total PSA ratio in the subgroup of patients with % free PSA<20. At 90% sensitivity (hK11/total PSA ratio of 0.05) the specificity is 54%. P value was determined by the Mann-Whitney U test

FIG. 3 shows receiver operating characteristic (ROC) curves for total PSA (tPSA), % free PSA and hK11/total PSA ratio, demonstrating the relative potential of each variable in the discrimination of BPH from CaP. AUC, area under the ROC curve; CI, confidence interval

FIG. 4 shows the percentage of potentially avoidable biopsies by various biochemical parameters. Eight % of BPH patients would have avoided biopsies by combining of % free PSA and the hK11/tPSA ratio (at 90% sensitivity).

FIG. 5 shows receiver operating characteristic (ROC) curves for total PSA, % free PSA/Total PSA ratio, and hK11/total PSA ratio, demonstrating the relative potential of each variable in the discrimination of BPH from CaP. AUC, area under the ROC curve; CI, confidence interval

FIG. 6 shows the distribution of hK11/tPSA ratio in BPH and CaP patients.

FIG. 7 shows the distribution of hK11/tPSA ratio in BPH and CaP patients where total PSA=4-10 μg/L.

FIG. 8 shows the distribution of hK11/tPSA ratio in BPH and CaP patients where total PSA=>10 μ/L.

FIG. 9 shows the distribution of hK11/tPSA ratio in BPH and CaP patients where total PSA=<4 μ/L.

FIG. 10 shows the distribution of hK11/tPSA ratio in BPH and CaP patients where total PSA=<17.8 μ/L (90% sensitivity).

### DETAILED DESCRIPTION OF THE INVENTION

The invention relates to newly discovered correlations between expression of kallikrein 11 in combination with PSA and prostate cancer. The methods described herein provide sensitive methods for detecting prostate cancer. The level of expression of kallikrein 11 and PSA correlates with the presence of prostate cancer. The invention is also based on the finding that kallikrein 11 levels and kallikrein11:PSA ration are significantly lower in prostate cancer patients then in benign prostatic hyperplasia.

Methods are provided for detecting the presence of prostate cancer in a sample, the absence of prostate cancer in a sample, assessing the histology of tissues associated with prostate cancer, and other characteristics of prostate cancer that are relevant to prevention, diagnosis, characterization, and therapy of prostate cancer in a patient. Methods are also provided for assessing the efficacy of one or more test agents for inhibiting kallikrein 11 and PSA that affect prostate cancer, assessing the efficacy of a therapy for prostate cancer, monitoring the progression of prostate cancer, selecting an agent or therapy for inhibiting prostate cancer, treating a patient afflicted with prostate cancer, inhibiting prostate cancer in a patient, and assessing the potential of a test compound to contribute to prostate cancer.

The invention particularly provides methods and kits for detecting prostate cancer and differentiating prostate cancer and benign prostatic hyperplasia in a subject by determining amounts of kallikrein 11 alone, or kallikrein 11 and PSA in samples from the subjects

Glossary

Samples that may be analyzed using the methods of the invention include those which are known or suspected to express kallikrein 11 and PSA or contain kallikrein 11 and PSA. The terms “sample”, “biological sample”, and the like mean a material known or suspected of expressing or containing kallikrein 11 and PSA, in particular kallikrein 11 and PSA associated with prostate cancer. The test sample can be used directly as obtained from the source or following a pretreatment to modify the character of the sample. The sample can be derived from any biological source, such as tissues, extracts, or cell cultures, including cells (e.g. tumor cells), cell lysates, and physiological fluids, such as, for example, whole blood, plasma, serum, saliva, ocular lens fluid, cerebral spinal fluid, sweat, urine, milk, ascites fluid, synovial fluid, peritoneal fluid and the like.

The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like. Proteins may be isolated from the samples and utilized in the methods of the invention.

In embodiments of the invention the sample is a mammalian tissue sample. In another embodiment the sample is a human physiological fluid. In a particular embodiment, the sample is human serum, seminal plasma, urine, or plasma, most preferably serum.

The terms “subject”, “individual” or “patient” refer to a warm-blooded animal such as a mammal, which is afflicted with or suspected of having or being pre-disposed to prostate cancer or condition as described herein. In particular, the terms refer to a human.

The term “kallikrein 11”, “kallikrein 11 polypeptide” or “kallikrein 11 protein” includes human kallikrein 11(“hK11”), in particular the native-sequence polypeptide, isoforms, chimeric polypeptides, all homologs, fragments, and precursors of human kallikrein 11. The amino acid sequence for native hK11 include the sequences of GenBank Accession Nos. BAA33404 and AB012917 and shown in SEQ ID NO. 1.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having at least about 70-80%, preferably at least about 85%, more preferably at least about 90%, most preferably at least about 95% amino acid sequence identity with a native-sequence polypeptide, in particular having at least 70-80%, 85%, 90%, 95% amino acid sequence identity to the sequences identified in the GenBank Accession Nos. BAA33404 and AB012917, and shown in SEQ ID NO. 1. Such variants include, for instance, polypeptides wherein one or more amino acid residues are added to, or deleted from, the N- or C-terminus of the full-length or mature sequences of SEQ ID NO: 1, including variants from other species, but excludes a native-sequence polypeptide.

An allelic variant may also be created by introducing substitutions, additions, or deletions into a nucleic acid encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which include fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a kallikrein polypeptide homolog, for example, the murine Kallikrein polypeptide.

The invention also includes polypeptides that are substantially identical to the sequences of GenBank Accession Nos. BAA33404 and AB012917 and shown in SEQ ID NO. 1 (e.g. at least about 45%, preferably 50%, 55%, 60%, 65%, 70%, 75%, 80%, 85%, 90%, 95%, 97%, 98%, or 99% sequence identity), and in particular polypeptides that retain the immunogenic activity of the corresponding native-sequence polypeptide.

Percent identity of two amino acid sequences, or of two nucleic acid sequences identified herein is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

Kallikrein 11 proteins include chimeric or fusion proteins. A “chimeric protein” or “fusion protein” comprises all or part (preferably biologically active) of a kallikrein 11 polypeptide operably linked to a heterologous polypeptide (i.e., a polypeptide other than a kallikrein 11 polypeptide). Within the fusion protein, the term “operably linked” is intended to indicate that a kallikrein 11 polypeptide and the heterologous polypeptide are fused in-frame to each other. The heterologous polypeptide can be fused to the N-terminus or C-terminus of a kallikrein 11 polypeptide. A useful fusion protein is a GST fusion protein in which a kallikrein 11polypeptide is fused to the C-terminus of GST sequences. Another example of a fusion protein is an immunoglobulin fusion protein in which all or part of a kallikrein 11 polypeptide is fused to sequences derived from a member of the immunoglobulin protein family. Chimeric and fusion proteins can be produced by standard recombinant DNA techniques.

Kallikrein polypeptides may be isolated from a variety of sources, such as from human tissue types or from another source, or prepared by recombinant or synthetic methods, or by any combination of these and similar techniques.

The terms “KLK11” or “KLK11 nucleic acid(s)” are intended to include DNA and RNA (e.g. mRNA) and can be either double stranded or single stranded. The terms include but are not limited to nucleic acids that encode a native-sequence polypeptide, a polypeptide variant including a portion of a kallikrein polypeptide, an isoform, precursor, and chimeric polypeptide. The nucleic acid sequences encoding native kallikrein polypeptides employed in the present invention include the nucleic acid sequences of GenBank Accession No. Accession Nos. AF164623 and AB012917 and in SEQ ID NO: 2 and 3, or fragments thereof.

Prostate specific antigen (PSA) is a 33-kDa glycosylated single chain serine protease (Lilja, J Clin Invest 1985; Watt et al. Proc Natl Acad Sci (USA) 1986). PSA has been predicted to be produced as an inactive zymogen (Lundwall et al. FEBS Lett 1987). Active PSA is secreted into the seminal plasma (Lilja, J Clin Invest 1985) and it is one of the most abundant proteins of the prostate (Lilja et al. The Prostate 1988; Dub et al. J Androl 1987). PSA is capable of forming complexes with serum protease inhibitors. Human serum contains high levels of α₁₋antichymotrypsin (ACT) and α₂-macroglobulin, both of which form complexes with PSA. About 70-95% of the PSA in serum that can be detected by immunoassay is complexed with ACT. About 30% of serum PSA does not form a complex with ACT. This non-complexed fraction of PSA contains an internal peptide bond cleavage at Lys145 which renders it inactive. PSA in a complex with a protease inhibitor is referred to “complexed PSA”, and PSA that is not complexed with a protease inhibitor is referred to herein as “free PSA”.

The term “total PSA” in reference to a sample refers to the total complexed PSA and free PSA in the sample.

“Statistically different levels” or “significant difference” in levels of markers in a patient sample compared to a control or standard (e.g. normal levels or levels in other samples from a patient) may represent levels that are higher or lower, in particular lower, than the standard error of the detection assay.

“Binding agent” refers to a substance such as a polypeptide or antibody that specifically binds to a kallikrein 11 or PSA. A substance “specifically binds” to kallikrein 11 or PSA if it reacts at a detectable level with a kallikrein 11 or PSA, and does not react detectably with peptides containing unrelated sequences or sequences of different polypeptides. Binding properties may be assessed using an ELISA, which may be readily performed by those skilled in the art (see for example, Newton et al, Develop. Dynamics 197: 1-13, 1993).

A binding agent may be a ribosome, with or without a peptide component, an RNA molecule, or a polypeptide. A binding agent may be a polypeptide that comprises a kallikrein 11 or PSA polypeptide sequence, a peptide variant thereof, or a non-peptide mimetic of such a sequence. By way of example a kallikrein 11 or PSA sequence may be a peptide portion of a kallikrein 11 or PSA that is capable of modulating a function mediated by the kallikrein 11 or PSA.

Antibodies for use in the present invention include but are not limited to monoclonal or polyclonal antibodies, immunologically active fragments (e.g. a Fab or (Fab)₂ fragments), antibody heavy chains, humanized antibodies, antibody light chains, genetically engineered single chain Fᵥ molecules (Ladner et al, U.S. Pat. No. 4,946,778), chimeric antibodies, for example, antibodies which contain the binding specificity of murine antibodies, but in which the remaining portions are of human origin, or derivatives, such as enzyme conjugates or labeled derivatives.

Antibodies including monoclonal and polyclonal antibodies, fragments and chimeras, may be prepared using methods known to those skilled in the art. Isolated native or recombinant kallikrein 11 and PSA may be utilized to prepare antibodies. See, for example, Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science. 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. Antibodies specific for kallikrein 11 and PSA may also be obtained from scientific or commercial sources.

In an embodiment of the invention, antibodies are reactive against kallikrein 11 and PSA if they bind with a Kₐ of greater than or equal to 10⁻⁷ M.

Methods for Detecting Kallikrein 11 and PSA

A variety of methods can be employed for the diagnostic and prognostic evaluation of prostate cancer involving kallikrein 11 and PSA, and the identification of subjects with a predisposition to such disorders. Such methods may, for example, utilize binding agents (e.g. antibodies) directed against kallikrein 11 and PSA, including peptide fragments. In particular, antibodies may be used, for example, for the detection of either an over- or an under-abundance of kallikrein 11 and PSA relative to a non-disorder state or the presence of a modified (e.g., less than full length) kallikrein 11 and PSA which correlates with a disorder state, or a progression toward a disorder state.

The invention also contemplates a method for detecting prostate cancer comprising producing a profile of levels of kallikrein 11 and PSA and other markers associated with prostate cancer in cells from a patient, and comparing the profile with a reference to identify a profile for the test cells indicative of disease.

The methods described herein may be used to evaluate the probability of the presence of malignant or pre-malignant cells, for example, in a group of cells freshly removed from a host. Such methods can be used to detect tumors, quantitate their growth, and help in the diagnosis and prognosis of disease. For example, lower levels of kallikrein 11 and PSA may be indicative of advanced disease, e.g. advanced prostate cancer. The methods can be used to detect the presence of cancer metastasis, as well as confirm the absence or removal of all tumor tissue following surgery, cancer chemotherapy, and/or radiation therapy. They can further be used to monitor cancer chemotherapy and tumor reappearance.

The methods described herein can be adapted for diagnosing and monitoring prostate cancer by detecting kallikrein 11 and PSA in biological samples from a subject. These applications require that the amount of kallikrein 11 and PSA quantitated in a sample from a subject being tested be compared to levels quantitated for another sample or an earlier sample from the subject, or levels quantitated for a control sample. Levels for control samples from healthy subjects or prostate cancer subjects may be established by prospective and/or retrospective statistical studies. Healthy subjects who have no clinically evident disease or abnormalities may be selected for statistical studies. Diagnosis may be made by a finding of statistically different levels of kallikrein 11 and PSA compared to a control sample or previous levels quantitated for the same subject.

Binding agents specific for kallikrein 11 and PSA may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of prostate cancer in a subject may be determined by (a) contacting a sample from the subject with binding agents for kallikrein 11 and PSA; (b) detecting in the sample levels of kallikrein 11 and PSA that bind to the binding agents; and (c) comparing the levels of kallikrein 11 and PSA with predetermined standards or cut-off values.

In particular embodiments, the binding agent is an antibody.

In an aspect, the invention provides a diagnostic method for monitoring or diagnosing prostate cancer in a subject by quantitating kallikrein 11 and PSA in a biological sample from the subject comprising reacting the sample with antibodies specific for kallikrein 11 and PSA, which are directly or indirectly labeled with detectable substances and detecting the detectable substances.

In an aspect of the invention, a method for detecting prostate cancer is provided comprising:
(a) obtaining a sample suspected of containing kallikrein 11 and PSA associated with prostate cancer;
(b) contacting said sample with antibodies that specifically bind kallikrein 11 and PSA under conditions effective to bind the antibodies and form complexes;
(c) measuring the amount of kallikrein 11 and PSA present in the sample by quantitating the amount of the complexes; and
(d) comparing the amount of kallikrein 11 and PSA present in the samples with the amount of kallikrein 11 and PSA in a control, wherein a change or significant difference in the amount of kallikrein 11 and PSA in the sample compared with the amount in the control is indicative of prostate cancer.

In an embodiment, the invention contemplates a method for monitoring the progression of prostate cancer in an individual, comprising:
(a) contacting antibodies which bind to kallikrein 11 and PSA with a sample from the individual so as to form complexes comprising the antibodies and kallikrein 11 or PSA in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of disease, disease stage, and/or progression of the cancer in said individual.

The amount of complexes may also be compared to a value representative of the amount of the complexes from an individual not at risk of, or afflicted with, prostate cancer at different stages. An decrease in complex formation may be indicative of advanced disease e.g. advanced prostate cancer, or an unfavourable prognosis.

Antibodies specifically reactive with kallikrein 11 and PSA, or derivatives, such as enzyme conjugates or labeled derivatives, may be used to detect kallikrein 11 and PSA proteins in various samples (e.g. biological materials). They may be used as diagnostic or prognostic reagents and they may be used to detect abnormalities in the level of kallikrein 11 and PSA expression, or abnormalities in the structure, and/or temporal, tissue, cellular, or subcellular location of kallikrein 11 and PSA. Antibodies may also be used to screen potentially therapeutic compounds in vitro to determine their effects on disorders (e.g. prostate cancer) involving a kallikrein 11 and PSA protein, and other conditions. In vitro immunoassays may also be used to assess or monitor the efficacy of particular therapies.

Antibodies may be used in any known immunoassays that rely on the binding interaction between antigenic determinants of kallikrein 11 and PSA protein and the antibodies. Immunoassay procedures for in vitro detection of antigens in fluid samples are also well known in the art [See for example, Paterson et al., Int. J. Can. 37:659 (1986) and Burchell et al., Int. J. Can. 34:763 (1984) for a general description of immunoassay procedures]. Qualitative and/or quantitative determinations of kallikrein 11 and PSA in a sample may be accomplished by competitive or non-competitive immunoassay procedures in either a direct or indirect format Detection of kallikrein 11 and PSA using antibodies can be done utilizing immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. These terms are well understood by those skilled in the art. A person skilled in the art will know, or can readily discern, other immunoassay formats without undue experimentation. The antibodies may be used to detect and quantify kallikrein 11 and PSA in a sample in order to diagnose and treat pathological states.

In particular, the antibodies may be used in immunohistochemical analyses, for example, at the cellular and sub-subcellular level, to detect kallikrein 11 and PSA proteins, to localize them to particular prostate tumor cells and tissues, and to specific subcellular locations, and to quantitate the level of expression.

Immunohistochemical methods for the detection of antigens in tissue samples are well known in the art. For example, immunohistochemical methods are described in Taylor, Arch. Pathol. Lab. Med. 102:112 (1978). Briefly, in the context of the present invention, a tissue sample obtained from a subject suspected of having a prostate-related problem is contacted with antibodies, preferably monoclonal antibodies recognizing kallikrein 11. The site at which the antibodies are bound is determined by selective staining of the sample by standard immunohistochemical procedures. The same procedure may be repeated on the same sample using other antibodies that recognize kallikrein 11. Alternatively, a sample may be contacted with antibodies against kallikrein 11 and antibodies against PSA simultaneously, provided that the antibodies are labeled differently or are able to bind to a different label. In one embodiment of the present invention, the tissue sample is obtained from the prostate of a patient. The prostate tissue sample may be a normal prostate tissue, a cancer prostate tissue or a benign prostatic hyperplasia tissue.

In a sandwich immunoassay of the invention mouse polyclonal/monoclonal antibodies specific for kallikrein 11 and PSA and rabbit polyclonal/monoclonal antibodies specific for kallikrein 11 and PSA are utilized.

Antibodies specific for kallikrein 11 and PSA may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

One of the ways an antibody can be detectably labeled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase, glucose-6-phosphate, staphylococcal nuclease, delta-5-steriod isomerase, yeast alcohol dehydrogenase, alpha-glycerophosphate, triose phosphate isomerase, asparaginase, glucose oxidase, and acetylcholine esterase.

For increased sensitivity in an immunoassay system a fluorescence-emitting metal atom such as Eu (europium) and other lanthanides can be used. These can be attached to the desired molecule by means of metal-chelating groups such as DTPA or EDTA.

A bioluminescent compound may also be used as a detectable substance. Bioluminescence is a type of chemiluminescence found in biological systems where a catalytic protein increases the efficiency of the chemiluminescent reaction. The presence of a bioluminescent molecule is determined by detecting the presence of luminescence. Examples of bioluminescent detectable substances are luciferin, luciferase and aequorin.

Indirect methods may also be employed in which the primary antigen-antibody reaction is amplified by the introduction of a second antibody, having specificity for the antibody reactive against kallikrein 11 and PSA. By way of example, if the antibody having specificity against kallikrein 11 and PSA is a rabbit IgG antibody, the second antibody may be goat anti-rabbit IgG, Fc fragment specific antibody labelled with a detectable substance as described herein.

Methods for conjugating or labelling the antibodies discussed above may be readily accomplished by one of ordinary skill in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,”Anal. Biochem. 171:1-32, 1988 re methods for conjugating or labelling the antibodies with enzyme or ligand binding partner).

Cytochemical techniques known in the art for localizing antigens using light and electron microscopy may be used to detect a kallikrein 11 and PSA protein. Generally, an antibody may be labeled with a detectable substance and a kallikrein 11 and PSA protein may be localised in tissues and cells based upon the presence of the detectable substance.

In the context of the methods of the invention, the sample, binding agents (e.g. antibodies) or kallikrein 11 and PSA may be immobilized on a carrier or support Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized material may be prepared by reacting the material with a suitable insoluble carrier using known chemical or physical methods, for example, cyanogen bromide coupling. Binding agents (e.g. antibodies) may be indirectly immobilized using second binding agents specific for the first binding agent. For example, mouse antibodies specific for kallikrein 11 and PSA may be immobilized using sheep anti-mouse IgG Fc fragment specific antibody coated on the carrier or support.

Where a radioactive label is used as a detectable substance, a kallikrein 11 and PSA protein may be localized by radioautography. The results of radioautography may be quantitated by determining the density of particles in the radioautographs by various optical methods, or by counting the grains.

Time-resolved fluorometry may be used to detect a fluorescent signal, label, or detectable substance. For example, the method described in Christopoulos T K and Diamandis E P Anal. Chem., 1992:64:342-346 may be used with a conventional time-resolved fluorometer.

According to an embodiment of the invention, an immunoassay for detecting kallikrein 11 in a biological sample comprises contacting an amount of a first agent that specifically binds to kallikrein 11 in the sample under a condition that allows the formation of a first complex comprising the first agent and kallikrein 11 and determining the presence or amount of the complex as a measure of the amount of the kallikrein 11 contained in the sample. In another embodiment, an immunoassay is provided for detecting kallikrein 11 and total PSA in a sample comprising the additional step of contacting an amount of a second agent which specifically binds to PSA with the sample under a condition that allows the formation of a second complex comprising the second agent and PSA, and determining the presence or amount of the second complex as a measure of the amount of the PSA contained in the sample. In an alternate method, a sample may be contacted by the first and the second agents simultaneously, provided that the agents are labeled differently or are capable of binding to different labels.

In accordance with an embodiment of the invention, a method is provided wherein kallikrein 11 and PSA antibodies are directly or indirectly labelled with enzymes, substrates for the enzymes are added wherein the substrates are selected so that the substrates, or a reaction product of an enzyme and substrate, form fluorescent complexes with a lanthanide metal (e.g. europium, terbium, samarium, and dysprosium, preferably europium and terbium). A lanthanide metal is added and kallikrein 11 and PSA are quantitated in the sample by measuring fluorescence of the fluorescent complexes. Enzymes are selected based on the ability of a substrate of the enzyme, or a reaction product of the enzyme and substrate, to complex with lanthanide metals such as europium and terbium. Suitable enzymes and substrates that provide fluorescent complexes are described in U.S. Pat. No. 5,3112,922 to Diamandis. Examples of suitable enzymes include alkaline phosphatase and β-galactosidase. Preferably, the enzyme is alkaline phosphatase.

Examples of enzymes and substrates for enzymes that provide such fluorescent complexes are described in U.S. Pat. No. 5,312,922 to Diamandis. By way of example, when the antibody is directly or indirectly labelled with alkaline phosphatase the substrate employed in the method may be 4-methylumbelliferyl phosphate, 5-fluorosalicyl phosphate, or diflunisal phosphate. The fluorescence intensity of the complexes is typically measured using a time-resolved fluorometer e.g. a CyberFluor 615 Imunoanalyzer (Nordion International, Kanata, Ontario).

Antibodies specific for kallikrein 11 and PSA may also be indirectly labelled with enzymes. For example, an antibody may be conjugated to one partner of a ligand binding pair, and the enzyme may be coupled to the other partner of the ligand binding pair. Representative examples include avidin-biotin, and riboflavin-riboflavin binding protein. In an embodiment, an antibody specific for the anti-kallikrein 11 and PSA antibody is labelled with an enzyme.

In accordance with an embodiment, the present invention provides means for determining kallikrein 11 and PSA in a serum sample by measuring kallikrein 11 and PSA by immunoassay. It will be evident to a skilled artisan that a variety of immunoassay methods can be used to measure kallikrein 11 and PSA in serum. In general, a kallikrein 11 and PSA immunoassay method may be competitive or noncompetitive. Competitive methods typically employ an immobilized or immobilizable antibody to kallikrein 11 and PSA (anti-kallikrein 11 and anti-PSA) and a labeled form of kallikrein 11 and PSA. Sample kallikrein 11 and PSA and labeled kallikrein 11 and PSA compete for binding to anti-kallikrein 11 and PSA. After separation of the resulting labeled kallikrein 11 and PSA that has become bound to anti-kallikrein 11 and PSA (bound fraction) from that which has remained unbound (unbound fraction), the amount of the label in either bound or unbound fraction is measured and may be correlated with the amount of kallikrein 11 and PSA in the test sample in any conventional manner, e.g., by comparison to a standard curve.

In an aspect, a non-competitive method is used for the determination of kallikrein 11 and PSA, with the most common method being the “sandwich” method. In this assay, two anti-kallikrein 11 and two PSA antibodies are employed. One of the anti-kallikrein 11 and one or the PSA antibodies is directly or indirectly labeled (sometimes referred to as the “detection antibody”) and the other is immobilized or immobilizable (sometimes referred to as the “capture antibody”). The capture and detection antibodies can be contacted simultaneously or sequentially with the test sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody may be separated from the liquid test mixture, and the label may be measured in at least a portion of the separated capture antibody phase or the remainder of the liquid test mixture. Generally it is measured in the capture antibody phase since it comprises kallikrein 11 and PSA bound by (“sandwiched” between) the capture and detection antibodies. In another embodiment, the label may be measured without separating the capture antibody and liquid test mixture.

In a typical two-site immunometric assay for kallikrein 11 and PSA, one or both of the capture and detection antibodies are polyclonal antibodies or one or both of the capture and detection antibodies are monoclonal antibodies (i.e. polyclonal/polyclonal, monoclonal/monoclonal, or monoclonal/polyclonal). The label used in the detection antibody can be selected from any of those known conventionally in the art. The label may be an enzyme or a chemiluminescent moiety, but it can also be a radioactive isotope, a fluorophor, a detectable ligand (e.g., detectable by a secondary binding by a labeled binding partner for the ligand), and the like. Preferably the antibody is labelled with an enzyme which is detected by adding a substrate that is selected so that a reaction product of the enzyme and substrate forms fluorescent complexes. The capture antibody may be selected so that it provides a means for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, the capture antibody may be immobilized using a species specific antibody for the capture antibody that is bound to the solid phase.

A particular sandwich immunoassay method of the invention employs antibodies reactive against kallikrein 11 and PSA, antibodies having specificity against antibodies reactive against kallikrein 11 and PSA labelled with an enzymatic label, and a fluorogenic substrate for the enzyme. In an embodiment, the enzyme is alkaline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(³⁺)-EDTA, which can be quantified by measuring the Tb³⁺ fluorescence in a time-resolved mode. Fluorescence intensity is measured using a time-resolved fluorometer as described herein.

The above-described immunoassay methods and formats are intended to be exemplary and are not limiting.

The invention also contemplates the methods described herein using multiple markers for prostate cancer. Therefore, the invention contemplates a method for anaylzing a biological sample for the presence of kallikrein 11 and PSA and other markers that are specific indicators of prostate cancer. The methods described herein may be modified by including reagents to detect the markers, or nucleic acids for the markers. The methods described herein may also include reagents to detect KLK11. Techniques for detecting nucleic acid such as polymerase chain reaction (PCR) and hybridization assays are well known in the art.

The use of kallikrein 11 alone, and kallikrein 11 in combination with PSA may lead to improved discrimination between BPH and prostate cancer. The mathematical combination of the amount of kallikrein 11 and PSA may be used as a serum marker or as an immunohistological marker to help distinguish prostate cancer from BPH. Alternatively, kallikrein 11 alone may be used as a serum marker or as an immunohistological marker for distinguishing BPH from prostate cancer. The term “mathematical combination” as used herein refers to any mathematical calculation of the amount of kallikrein 11 and PSA. In an aspect of the invention, the mathematical combination is a ratio. The ratio of kallikrein 11 and PSA in a sample may be determined by comparing the amount of kallikrein 11 to the amount of total PSA in the sample. Thus, the mathematical combination may be the ratio of kallikrein 11: total PSA or the inverse thereof.

The mathematical combination may be compared to the mathematical combination of a standard in order to determine the presence of BPH or prostate cancer in the subject. In a particular embodiment of the invention a kallikrein 11: total PSA ratio greater than 0.02, 0.03, 0.04, 0.05, 0.06, 0.07, 0.08, 0.09, 0.10, 0.11, or 0.12 is potentially indicative of BPH while a kallikrein 11: total PSA ratio less than 0.02, 0.03, 0.04, 0.05, 0.06, 0.07, 0.08, 0.09, 0.10, 0.11, or 0.12 is potentially indicative of prostate cancer. In a more particular embodiment, a kallikrein 11: total PSA ratio greater than 0.05 is potentially indicative of BPH while a kallikrein 11: total PSA ratio less than 0.05 is potentially indicative of prostate cancer.

Accordingly, another aspect of the present invention provides a method for determining the presence of BPH or prostate cancer in a subject comprising the steps of:
(a) providing a first binding agent that specifically binds to kallikrein 11;
(b) providing a second binding agent that specifically binds to PSA;
(c) contacting the first agent and the second agent with a sample from the subject under a condition that allows the formation of a first complex comprising the first agent and kallikrein 11 and a second complex comprising the second agent and the PSA;
(d) detecting or determining the presence or amount of the first and second complexes;
(e) mathematically combining the amount of the first and the second complexes or the amount of kallikrein 11; and
(f) relating the combination to the presence of BPH or prostate cancer in the subject.

In accordance with embodiments of the present invention, the agents comprise antibodies. Preferably, the first and the second agents are directly or indirectly labeled with different detectable substances to form respective complexes that may be detected separately. Alternatively, the sample may be contacted with one agent first so that either kallikrein 11 may be detected first, then the same sample may be contacted with another agent in order to detect PSA.

In one embodiment of the present invention, the sample used in the diagnostic method may be a sample of human physiological fluid such as, but not limited to, serum, seminal plasma, urine and plasma, preferably serum. In another embodiment, the sample may be tissue specimen from the prostate of a patient.

In a particular embodiment, the combination is a ratio of the first complex to the second complex, or the inverse thereof.

In particular methods of the invention, patients with BPH or prostate cancer can be identified in patients with total PSA between about 4-10 ng/ml.

In particular methods of the invention, patients with BPH or prostate cancer can be identified in patients with total PSA less than about 4 ng/ml.

The methods of the invention may utilize commercially available kits for detecting or quantifying PSA in a sample. For example the Immulite PSA (Diagnostic Products Corporation, Cal, USA) assay may be used to measure PSA in a sample.

Since kallikrein 11 alone may be used as a serum marker or as an immunohistological marker for distinguishing BPH from prostate cancer, the present invention also provides a diagnostic method for distinguishing BPH from prostate cancer by detecting and determining the amount of kallikrein 11 in a sample. The amount of kallikrein 11 may be determined in tissue samples by immunohistochemical methods and/or in patient fluid samples by in vitro immunoassay procedures described herein.

Computer Systems

Computer readable media comprising kallikrein 11 and PSA and optionally other markers of prostate cancer is also provided. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on kallikrein 11 and PSA and optionally other prostate cancer markers.

A variety of data processor programs and formats can be used to store information on kallikrein 11 and PSA and other prostate cancer markers on computer readable medium. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has prostate cancer or a pre-disposition to prostate cancer, comprising determining the presence, absence, or amount of kallikrein 11 and PSA and optionally other prostate cancer markers, and based on the presence, absence or amount of kallikrein 11 and PSA and optionally other markers, determining whether the patient has prostate cancer, BPH, or a pre-disposition to prostate cancer, and optionally recommending treatment for the condition.

The invention also provides in an electronic system and/or in a network, a method for determining whether a subject has prostate cancer or a pre-disposition to prostate cancer, comprising determining the presence, absence, or amount of kallikrein 11 and PSA and optionally other prostate cancer markers, and based on the presence, absence, or amount of the kallikrein 11 and PSA and optionally other markers, determining whether the subject has prostate cancer, BPH, or a pre-disposition to prostate cancer, and optionally recommending treatment for the condition.

The invention further provides in a network, a method for determining whether a subject has prostate cancer or a pre-disposition to prostate cancer comprising: (a) receiving phenotypic information on the subject and information on kallikrein 11 and PSA and optionally other prostate cancer markers associated with samples from the subject; (b) acquiring information from the network corresponding to the kallikrein 11 and PSA and optionally other markers; and (c) based on the phenotypic information and information on the kallikrein 11 and PSA and optionally other markers determining whether the subject has prostate cancer, BPH or a pre-disposition to prostate cancer; and (d) optionally recommending treatment for the condition.

The invention still further provides a system for identifying selected records that identify a prostate cancer cell or tissue. A system of the invention generally comprises a digital computer; a database server coupled to the computer; a database coupled to the database server having data stored therein, the data comprising records of data comprising kallikrein 11 and PSA and optionally other prostate cancer markers, or nucleic acids encoding same, and a code mechanism for applying queries based upon a desired selection criteria to the data file in the database to produce reports of records which match the desired selection criteria.

In an aspect of the invention a method is provided for detecting prostate cancer tissue or cells using a computer having a processor, memory, display, and input/output devices, the method comprising the steps of:
(a) creating records of kallikrein 11 and PSA including mathematical combinations thereof, and optionally other prostate cancer markers isolated from a sample suspected of containing prostate cancer cells or tissue;
(b) providing a database comprising records of data comprising kallikrein 11 and PSA including mathematical combinations thereof, and optionally other prostate cancer markers; and
(c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from cells or tissue that are prostate cancer cells or tissue.

The invention contemplates a business method for determining whether a subject has prostate cancer or a pre-disposition to prostate cancer comprising: (a) receiving phenotypic information on the subject and information on kallikrein 11 and PSA and optionally other prostate cancer markers associated with samples from the subject; (b) acquiring information from a network corresponding to kallikrein 11 and PSA and optionally other markers; and (c) based on the phenotypic information, information on kallikrein 11 and PSA and optionally other markers, and acquired information, determining whether the subject has prostate cancer or a pre-disposition to prostate cancer; and (d) optionally recommending treatment for the prostate cancer or pre-condition.

In an aspect of the invention, the computer systems, components, and methods described herein are used to monitor disease or determine the stage of disease.

Screening Methods

The invention also contemplates methods for evaluating test agents or compounds for their ability to inhibit prostate cancer or potentially contribute to prostate cancer. Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The agents or compounds may be endogenous physiological compounds or natural or synthetic compounds.

The invention provides a method for assessing the potential efficacy of a test agent for inhibiting prostate cancer in a patient, the method comprising comparing:
(a) levels of kallikrein 11 and PSA including mathematical combinations thereof, and optionally other prostate cancer markers in a first sample obtained from a patient and exposed to the test agent; and
(b) levels of kallikrein 11 and PSA including mathematical combinations thereof, and optionally other markers in a second sample obtained from the patient, wherein the sample is not exposed to the test agent, wherein a significant difference in the levels of expression of kallikrein 11 and PSA including mathematical combinations thereof, and optionally the other markers in the first sample, relative to the second sample, is an indication that the test agent is potentially efficacious for inhibiting prostate cancer in the patient.

The first and second samples may be portions of a single sample obtained from a patient or portions of pooled samples obtained from a patient.

In an embodiment, the levels of expression of kallikrein 11 and PSA including mathematical combinations thereof, in the first sample are significantly higher relative to the second sample.

In an aspect, the invention provides a method of selecting an agent for inhibiting prostate cancer in a patient comprising:
(a) obtaining a sample from the patient;
(b) separately maintaining aliquots of the sample in the presence of a plurality of test agents;
(c) comparing kallikrein 11 and PSA including mathematical combinations thereof and optionally other prostate cancer markers, in each of the aliquots; and
(d) selecting one of the test agents which alters the levels of kallikrein 11 and PSA including mathematical combinations thereof and optionally other prostate cancer markers in the aliquot containing that test agent, relative to other test agents.

In an embodiment, the levels of kallikrein 11 and PSA including mathematical combinations thereof are significantly higher in the presence of the selected test agent.

Still another aspect of the present invention provides a method of conducting a drug discovery business comprising:
(a) providing one or more methods or assay systems for identifying agents that inhibit prostate cancer in a patient;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the potential of a test compound to contribute to prostate cancer comprising:
(a) maintaining separate aliquots of cells or tissues from a patient with prostate cancer in the presence and absence of the test compound; and
(b) comparing kallikrein 11 and PSA including mathematical combinations thereof and optionally other prostate cancer markers in each of the aliquots.

A significant difference between the levels of the markers in the aliquot maintained in the presence of (or exposed to) the test compound relative to the aliquot maintained in the absence of the test compound, indicates that the test compound possesses the potential to contribute to prostate cancer. In an embodiment, the levels of kallikrein 11 and PSA including mathematical combinations thereof are lower in the presence of the test compound.

In an aspect the invention provides a method of inhibiting prostate cancer in a patient, the method comprising (a) obtaining a sample comprising cells affected by prostate cancer from the patient; (b) separately maintaining aliquots of the sample in the presence of a plurality of test agents; (c) comparing levels of kallikrein 11 and PSA or a mathematical combination thereof in each of the aliquots; and (d) administering to the patient at least one of the test agents which alters kallikrein 11 and PSA or a mathematical combination thereof in the aliquot containing that test agent, relative to other test agents.

Kits

The invention contemplates kits for carrying out the methods of the invention. Such kits typically comprise two or more components required for performing a diagnostic assay. Components include but are not limited to compounds, reagents, containers, and/or equipment.

The methods described herein may be performed by utilizing pre-packaged diagnostic kits comprising at least binding agents (e.g. antibodies) described herein, which may be conveniently used, e.g. in clinical settings, to screen and diagnose patients, and to screen and identify those individuals afflicted with prostate cancer or BPH or exhibiting a predisposition to such condition.

In an embodiment, a container with a kit comprises binding agents as described herein. By way of example, the kit may contain antibodies specific for kallikrein 11 and PSA and optionally other prostate cancer markers, antibodies against the antibodies labelled with enzymes, and substrates for the enzymes. The kit may also contain microtiter plate wells, standards, assay diluent, wash buffer, adhesive plate covers, and/or instructions for carrying out a method of the invention using the kit.

In an aspect of the invention, the kit includes antibodies or antibody fragments which bind specifically to epitopes of kallikrein 11 and PSA and optionally other prostate cancer markers, and means for detecting binding of the antibodies to epitopes associated with prostate cancer, either as concentrates (including lyophilized compositions), which may be further diluted prior to use or at the concentration of use, where the vials may include one or more dosages. Where the kits are intended for in vivo use, single dosages may be provided in sterilized containers, having the desired amount and concentration of agents. Containers that provide a formulation for direct use, usually do not require other reagents.

In particular, the invention provides a kit for determining the presence of BPH or prostate comprising a known amount of a first binding agent that specifically binds to kallikrein 11 wherein the first binding agent comprises a detectable substance, or it binds directly or indirectly to a detectable substance.

Another aspect of the present invention also provides a kit for determining the presence of BPH or prostate cancer in a sample. The kit includes:
(a) a known amount of a first agent which specifically binds to kallikrein 11, and
(b) a known amount of a second agent which specifically binds to a PSA,
   wherein the first and the second agents, respectively, comprise a detectable label or bind to a detectable label.

The agents may be antibodies, particularly monoclonal antibodies. Preferably, the first and the second agents, respectively, comprise a different detectable substance or bind to a different detectable substance.

The reagents suitable for applying the screening methods of the invention to evaluate compounds may be packaged into convenient kits described herein providing the necessary materials packaged into suitable containers.

Thus, the invention relates to a kit for assessing the suitability of each of a plurality of test compounds for inhibiting prostate cancer in a patient The kit comprises reagents for assessing kallikrein 11 and PSA and optionally a plurality of test agents or compounds.

The invention contemplates a kit for assessing the presence of prostate cancer cells, wherein the kit comprises antibodies specific for kallikrein 11 and PSA, and optionally antibodies specific for other markers associated with prostate cancer.

Additionally the invention provides a kit for assessing the potential of a test compound to contribute to prostate cancer. The kit comprises prostate cancer cells and reagents for assessing kallikrein 11 and PSA, and optionally other markers associated with endocrine cancer.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLE 1

hK11, total PSA and % free PSA, was analyzed in a total of 150 serum samples from men with histologically confirmed benign prostatic hyperplasia (BPH; N=64) or prostatic cancer (CaP; N=86). Total and free PSA levels were measured by the Immulite PSA assay and hK11 levels were measured by an immunofluorometric assay. Serum hK11 levels and the hK11/total PSA ratio were both significantly lower in CaP patients than in BPH. In the subgroup of patients with % free PSA less than 20, an additional 54% of BPH patients could have avoided biopsies using the hK11/total PSA ratio. ROC curve analysis demonstrated that the hK11/total PSA ratio (AUC=0.81) and % free PSA (AUC=0.82) were much stronger predictors of prostate cancer than total PSA (AUC=0.69). Thus, the hK11/total PSA ratio is a useful tumor marker for prostate cancer and may be combined with % free PSA to further significantly reduce the number of unnecessary prostatic biopsies.

Materials and Methods

Study Population

Included in this study were serum samples from 150 male patients, 64 with benign prostate hyperplasia (BPH) (median age 65) and 86 with prostate cancer (CaP) (median age 62), all histologically confirmed by biopsy. All patients were from the Department of Urology at University Hospital Charité, Humboldt University, Berlin, Germany. Total and % free PSA, measured by the Immulite method (Diagnostic Products Crop., San Diego, Calif.) were available for all samples. All sera were stored at −80° C. until use.

The samples were collected with informed consent and the study was approved by the Institutional Review Board of the University Hospital Charité, Humboldt University, Berlin, Germany.

Production of Monoclonal and Polyclonal Antibodies Against hK11

Purified recombinant hK11 protein was used to immunize rabbits and mice, to raise polyclonal and monoclonal antibodies as described elsewhere¹¹. hK11 was produced by expression in Pichia Pastoris (Invitrogen) and purified by SP Sepharose cation exchange column and reverse phase liquid chromatography.

Development of Immunofluorometric Assay for hK11

In this study, the hK11 assay described previously was used, with minor modifications¹¹. In brief, sheep anti-mouse IgG (Jackson Immunoresearch) diluted in coating buffer (containing 50 mM Tris, pH 7.8) was dispensed into a 96-well white polystyrene microtiter plate (500 ng/100 μl/well) and incubated overnight at room temperature. The plates were then washed three times with washing buffer that contains 9 g/L NaCl and 0.5 g/L Tween 20 in 10 mM Tris, pH 7.8. One hundred μl of anti-hK11 monoclonal antibody (100 ng) diluted in 6% BSA were added to each well and incubated with orbital shaking for 2 hours at room temperature. The plates were washed 6 times with wash buffer. Fifty μl of hK 11 calibrators (recombinant hK11 in 6% BSA) or samples were applied into each well along with 50 μl of 6% BSA. The plates were incubated for 2 hours on an orbital shaker. After this step, the previously described procedure was used¹¹. All samples were analyzed in triplicate.

Statistical Analysis

The analysis of differences between measured or calculated parameters, in the two groups, were performed with the non-parametric Mann-Whitney U test. Relationships between different variables were assessed by Spearman correlation coefficient. Receiver operating characteristic (ROC) curves were constructed for total PSA, % free PSA and hK11/total PSA ratio, by plotting sensitivity versus (1-specificity) and the area under the ROC curves (AUC) were analyzed by the Hanley and McNeil method.

Results

hK11 levels were measured in 150 serum samples from 86 patients with prostate cancer and 64 patients with benign prostate hyperplasia with known total and % free PSA values. Descriptive statistics are summarized in Tables 1 and 2.

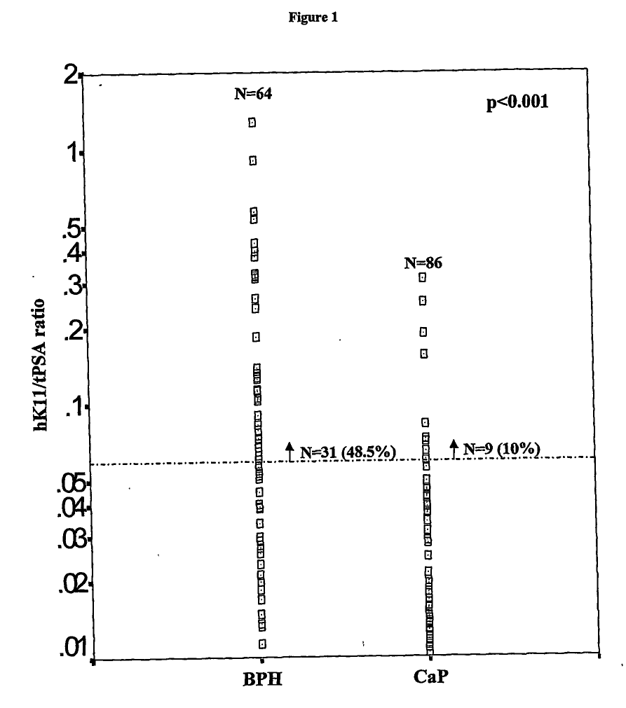

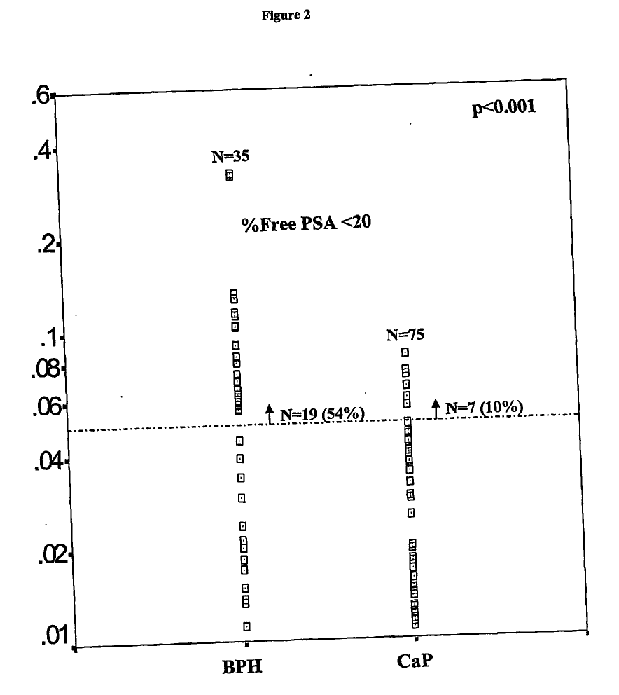

Total PSA values ranged from 0.17 to 26.2 μg/L in BPH patients, with a mean±SE of 5.61±0.63 μg/L and from 0.35 to 48.0 μg/L in CaP patients, with the mean±SE of 9.34:0.82 μg/L. Percent free PSA levels ranged from 0.85 to 64.0 (mean±SE=22.3±0.16) and from 1.9 to 47.9 (mean±SE=10.9±0.8) in patients with BPH and CaP, respectively. hK11 concentrations ranged from 0.0 to 2.33 μg/L (mean±SE of 0.41±0.05 μg/L) in BPH patients and from 0.0 to 0.72 μg/L (mean±SE of 0.15±0.02 μg/L) in CaP patients. The distributions of hK11 between the two groups were significantly different (p<0.001). The mean value±SE of hK11/total PSA ratio was 0.14±0.03 (range 0.00-1.28) in patients with BPH and 0.028±0.005 (range 0.00-0.31) in patients with CaP. The distribution of hK1/total PSA ratio was significantly lower in CaP patients than in BPH patients (p<0.001). At 90% sensitivity (hK11/total PSA ratio of 0.06) the specificity is 48.5% (FIG. 1). Furthermore, the hK11/total PSA ratio was analyzed in the subgroup of patients with % free PSA<20 (these patients would have undergone biopsy based on % free PSA). At a cutoff-point of 0.05(90% sensitivity), specificity was 54%. Nineteen of these 35 patients could have avoided biopsy based on this criterion (FIG. 2).

A weak positive correlation was found between hK11 and total PSA levels in the group of BPH patients (Spearman correlation coefficient rₛ=0.29, p=0.017). However, in CaP patients, a significant correlation between hK11 and other variables was not observed.

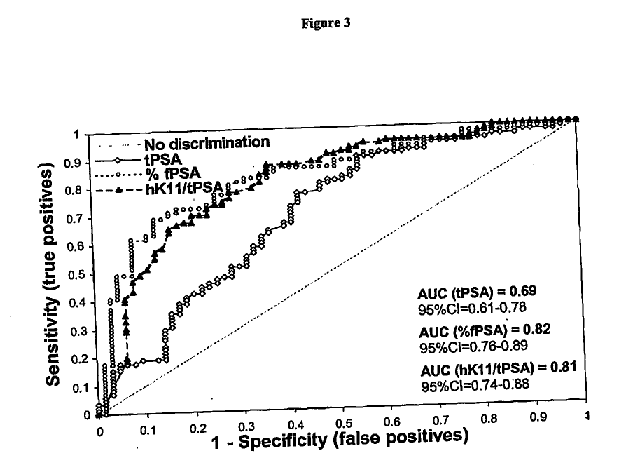

The discriminatory value of the hK11/total PSA ratio was investigated in relation to the total PSA and % free PSA values, by ROC curve analysis. The hK11/total PSA ratio, overall, had about the same discriminatory potential as % free PSA (AUC=0.81, 95% CI=0.74-0.88 for the hK 11/total PSA ratio and AUC=0.82, 95% CI=0.76-0.89 for the % free PSA) (FIG. 3).

Discussion

PSA is widely used as a tumor marker for prostate cancer, but the discrimination between BPH and CaP with this test is not possible due to the lack of specificity. It has been reported that PSA molecular forms, including free PSA, can contribute to better discrimination^{4,12-14}. hK2 is also useful for the differential diagnosis for prostate cancer in the PSA range of 2.5 to 10 μg/L^{4,15} .

Recently, an immunoassay was developed for hK11 and relatively large amounts of hK11 was found in seminal plasma and prostatic tissues¹¹.

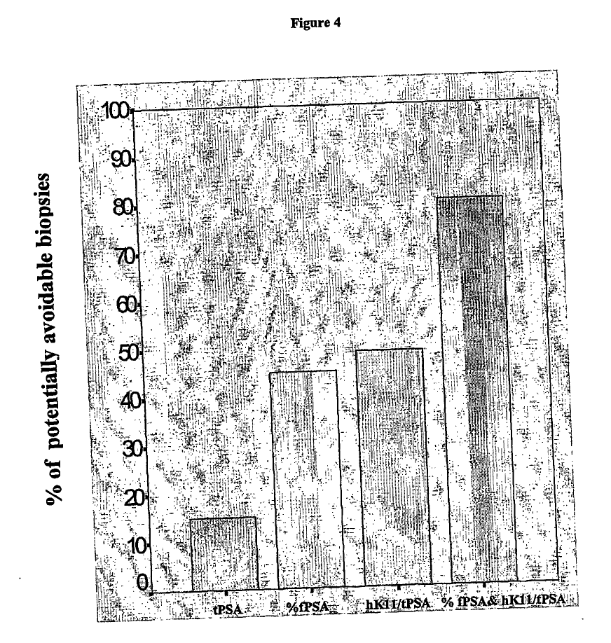

In this study, it is demonstrated for the first time that hK11 levels in serum are significantly lower in CaP patients than in BPH patients. The hK11/total PSA ratio is also significantly lower in CaP than in BPH. Using the hK11/total PSA ratio, approximately 50% of biopsies could be avoided that could have not been avoided by the % free PSA test (% free PSA<20). These results demonstrate for the first time that the combination of % free PSA and the hK11/total PSA ratio could contribute to better discrimination between BPH and CaP patients. Out of 64 patients with BPH, and at 90% sensitivity, 29 patients (45%) would have avoided biopsy by % free PSA testing (>20% free PSA). For the remaining 35 patients, another 19 could have avoided biopsy by the hK11/total PSA ratio (>0.05). When the two tests are combined, 48 BPH patients (80%) would have avoided biopsy at about 90% sensitivity (FIG. 4).

ROC curve analysis demonstrated that the hK11/total PSA ratio had about the same discriminatory value as % free PSA, suggesting that hK11 could be an additional marker for prostate cancer.

Conclusions

The potential clinical utility of serum hK11 analysis for the differential diagnosis of prostate cancer was investigated and it was determined that the hK11/total PSA ratio could be a useful novel tumor marker.

### EXAMPLE 2

Human kallikrein 11, total PSA, and % free PSA were analyzed using the procedure described in Example 1 in serum samples from 405 male patients, 247 with benign prostatic hyperplasia (median age 66) and 157 with prostate cancer (median age 64), histologically confirmed by biopsy in all cases. Descriptive statistics are described in Table 3. The logistic regression analysis of BPH and CaP patients for predicting the presence of prostate cancer is shown in Table 4. A comparison of the sensitivity and specificity at selected cut-off points of the study variables is shown in Table 5, and a comparison of the sensitivity and specificity at selected cut-off points in subgoups according to total PSA value is shown in Table 6. Table 7 shows serum hK11 concentration classified by tumor grade, stage of disease, and Gleasson score.

A positive correlation was found between hK11 and patient age in the group of patients with benign prostatic hyperplasia (Spearman correlation coefficient rₛ=0.242, p<0.001) as well as in the group of patients with prostate cancer (Spearman correlation coefficient rₛ=0.208, p=0.009).

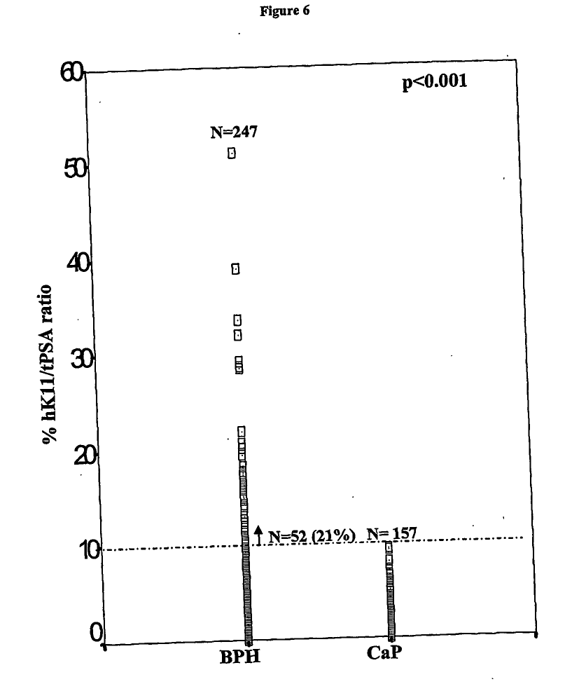

The distribution of hK1/total PSA ratio was significantly lower in CaP patients than in BPH patients (p<0.001) (FIG. 6).

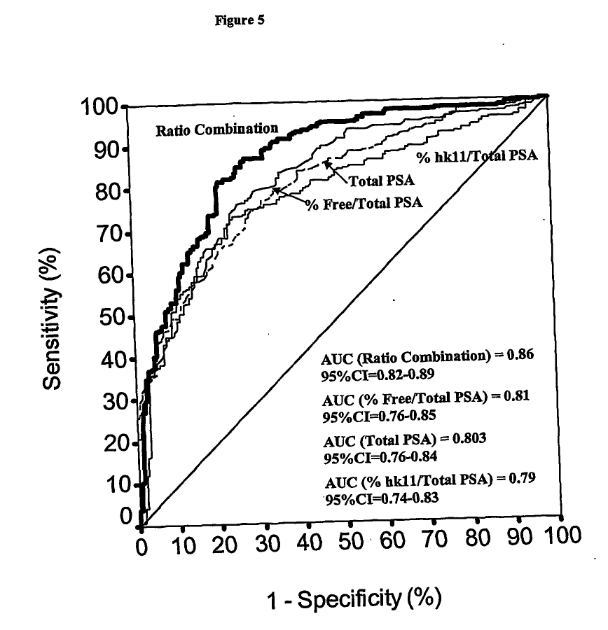

The discriminatory value of the hK11/total PSA ratio was investigated in relation to the total PSA and % free PSA values, by ROC curve analysis (FIG. 5). The hK11/total PSA ratio had about the same discriminatory potential as Total PSA.

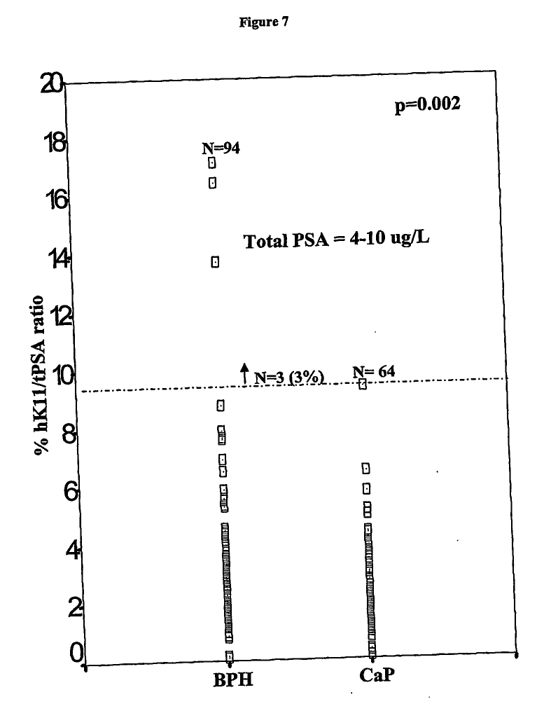

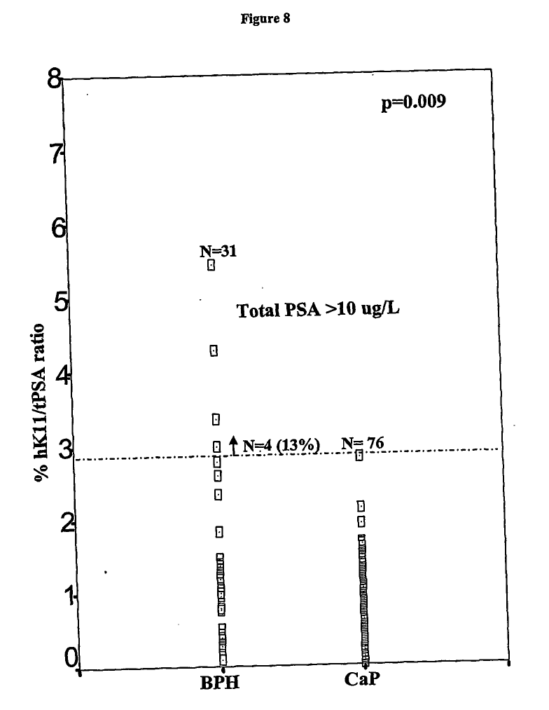

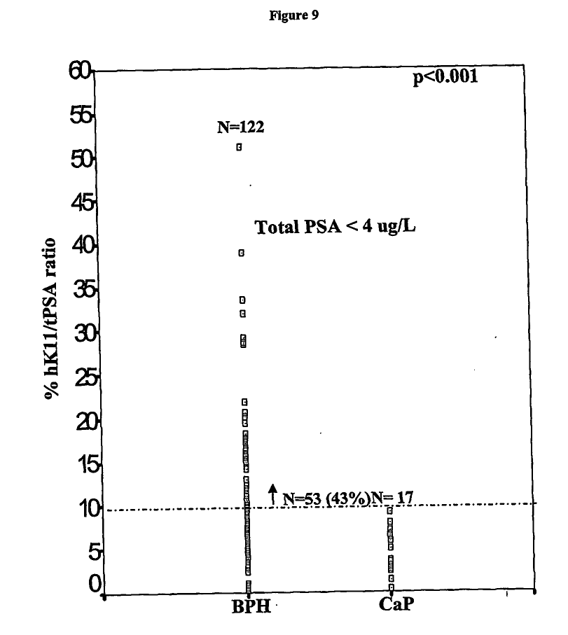

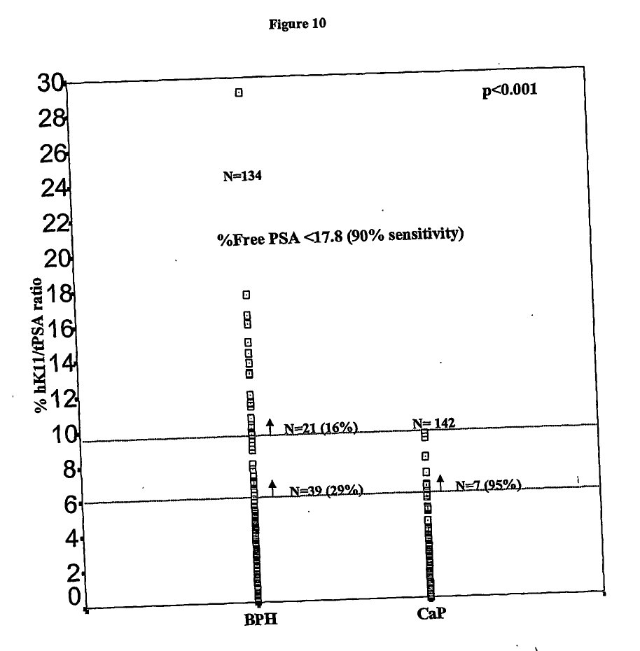

The hK11/total PSA ratio was analyzed in the subgroup of patients with total PSA=4-10 μg/L, total PSA>10 μg/L, total PSA<4 μg/L and % free PSA<17.8 (FIGS. 7 to 10). The ratio of hK11/total PSA was also shown to differentiate BPH from prostate cancer in a population of patients with total PSA less than 4 ng/ml (FIG. 9). In the range of 4-10 ng/ml total PSA, the combination of kallikrein 11, total PSA, and % free PSA was especially useful for differentiating BPH from prostate cancer.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the domains, cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Below full citations are set out for the references referred to in the specification.

TABLE 1
Descriptive statistics of various variables in serum of 64 BPH patients

| | | | Percentiles | | | | |
|---|---|---|---|---|---|---|---|
| | | | 10 | 25 | 50 | 75 | 90 |
| Variables | Mean ± SE^{a} | Range | (median) | | | | |
| hK11 (μg/L) | 0.41 ± 0.05 | 0.00-2.33 | 0.06 | 0.11 | 0.24 | 0.62 | 0.95 |
| Total PSA (μg/L) | 5.61 ± 0.63 | 0.17-26.2 | 1.05 | 1.63 | 4.17 | 8.02 | 12.95 |
| Free PSA (μg/L) | 1.22 ± 0.26 | 0.04-15.0 | 0.22 | 0.39 | 0.64 | 1.41 | 2.01 |
| % Free/Total PSA | 22.3 ± 0.1.56 | 0.85-64.0 | 10.0 | 13.4 | 18.5 | 28.2 | 38.4 |
| hK11/tPSA | 0.14 ± 0.03 | 0.00-1.28 | 0.013 | 0.029 | 0.063 | 0.13 | 0.39 |

^{a}Standard error

TABLE 2
Descriptive statistics of various variables in serum of 86 CaP patients

| | | | Percentiles | | | | |
|---|---|---|---|---|---|---|---|
| | | | 10 | 25 | 50 | 75 | 90 |
| Variables | Mean ± SE^{a} | Range | (median) | | | | |
| hK11 (μg/L) | 0.15 ± 0.02 | 0.00-0.72 | 0.00 | 0.05 | 0.08 | 0.25 | 0.38 |
| Total PSA (μg/L) | 9.34 ± 0.82 | 0.35-48.0 | 2.86 | 4.79 | 7.23 | 11.50 | 18.72 |
| Free PSA (μg/L) | 1.03 ± 0.26 | 0.10-23.0 | 0.27 | 0.42 | 0.61 | 0.97 | 1.55 |
| % Free/Total PSA | 10.9 ± 0.8 | 1.9-47.9 | 3.9 | 6.6 | 8.6 | 13.3 | 21.7 |
| hK11/tPSA | 0.028 ± 0.005 | 0.00-0.31 | 0.00 | 0.005 | 0.014 | 0.034 | 0.061 |

^{a}Standard error

TABLE 3
Descriptive statistics of various variables in serum of BPH and CaP patients

| Percentiles | | | | |
|---|---|---|---|---|
| 10 | 25 | 50 | 75 | 90 |

| | | | |
|---|---|---|---|
| Variables | Mean ± SE^{a} | Range | (median) |

| | | | | | | | |
|---|---|---|---|---|---|---|---|
| BPH (N = 247) | | | | | | | |
| HK11 (μg/L) | 0.19 ± 0.008 | 0.010^{b}-1.12 | 0.062 | 0.11 | 0.17 | 0.22 | 0.32 |
| Total PSA (μg/L) | 5.37 ± 0.42 | 0.58-82.9 | 1.18 | 1.90 | 4.00 | 6.50 | 11.62 |
| % Free/Total PSA | 17.8 ± 0.58 | 2.1-80.0 | 6.1 | 11.7 | 17.1 | 22.3 | 27.8 |
| % hK11/tPSA | 6.53 ± 0.43 | 0.085-51.14 | 0.84 | 2.03 | 4.44 | 8.80 | 15.23 |
| CaP (N = 157) | | | | | | | |
| HK11 (μg/L) | 0.14 ± 0.006 | 0.010^{b}-0.46 | 0.056 | 0.091 | 0.14 | 0.18 | 0.24 |
| Total PSA (μg/L) | 13.24 ± 1.07 | 0.61-90.7 | 3.85 | 5.72 | 9.50 | 14.87 | 25.25 |
| % Free/Total PSA | 9.87 ± 0.53 | 1.90-56.6 | 4.05 | 56.62 | 8.35 | 11.87 | 17.90 |
| % hK11/tPSA | 1.90 ± 0.14 | 0.02-9.38 | 0.31 | 0.73 | 1.30 | 2.32 | 4.33 |

^{a}Standard error
^{b}Half of the assay detection limit

TABLE 4
Logistic regression analysis of BPH and CaP patients for
predicting the presence of prostate cancer.

| | Univariate | | Multivariate | |
|---|---|---|---|---|
| | Analysis | | analysis | |
| | Crude | | Crude | |
| Covariate | Odds Ratio | p-value* | Odds Ratio | p-value* |
| Total PSA | 1.17 | <0.001 | 1.05 | 0.031 |
| % Free/Total PSA | 0.85 | <0.001 | 0.87 | 0.001 |
| Ratio | | | | |
| % hK11/Total PSA | 0.66 | <0.001 | 0.76 | <0.001 |
| Ratio | | | | |

*Test for trend

TABLE 5
Comparison of sensitivity and specificity at selected cut-off points
of the study variables

| Parameter | Cut-off | Specificity (%) | Sensitivity (%) |
|---|---|---|---|
| All Patients (N = 404) | | | |
| Total PSA | 14.92 | 95 | 25 |
| | 11.62 | 90 | 40 |
| | 3.86 | 49 | 90 |
| | 2.89 | 38 | 95 |
| % Free/Total PSA | 5.95 | 95 | 30 |
| | 8.35 | 90 | 51 |
| | 17.8 | 46 | 90 |
| | 19.2 | 39 | 95 |
| % hK11/Total PSA | 0.49 | 95 | 19 |
| | 0.93 | 90 | 33 |
| | 3.99 | 54 | 90 |
| | 5.97 | 38 | 95 |
| Ratio Combination^{a} | 1.50 | 95 | 54 |
| | 1.82 | 90 | 67 |
| | 3.19 | 56 | 90 |
| | 3.69 | 46 | 95 |

^{a}Ratio Combination function = 0.142 * % Free/Total PSA + 0.213 * % hK11/Total PSA as it was formulated from the logistic regression analysis.

TABLE 6
Comparison of sensitivity and specificity at selected
cut-off points in subgroups according to
total PSA value

| Parameter | Cut-off | Specificity (%) | Sensitivity (%) |
|---|---|---|---|
| Total PSA <4 μg/L (N = 139) | | | |
| % Free/Total PSA | 7.05 | 95 | 6 |
| | 8.85 | 90 | 18 |
| | 19.5 | 45 | 90 |
| | 20.0 | 44 | 95 |
| % hK11/Total PSA | 0.73 | 95 | 6 |
| | 2.92 | 90 | 35 |
| | 7.52 | 57 | 90 |
| | 9.19 | 43 | 95 |
| Ratio Combination^{a} | 2.12 | 95 | 18 |
| | 2.53 | 90 | 31 |
| | 4.12 | 58 | 90 |
| | 4.31 | 56 | 95 |
| Total PSA 4-10 μg/L | | | |
| (N = 158) | | | |
| % Free/Total PSA | 5.55 | 95 | 25 |
| | 8.35 | 90 | 53 |
| | 15.1 | 52 | 90 |
| | 18.2 | 42 | 95 |
| % hK11/Total PSA | 0.79 | 95 | 10 |
| | 1.25 | 90 | 22 |
| | 3.99 | 28 | 90 |
| | 5.24 | 16 | 95 |
| Ratio Combination^{a} | 1.61 | 95 | 47 |
| | 1.77 | 90 | 58 |
| | 2.71 | 56 | 90 |
| | 3.13 | 48 | 95 |
| Total PSA >10 μg/L | | | |
| (N = 107) | | | |
| % Free/Total PSA | 4.65 | 95 | 20 |
| | 6.35 | 90 | 40 |
| | 14.6 | 39 | 90 |
| | 25.8 | 10 | 95 |
| % hK11/Total PSA | 0.22 | 95 | 13 |
| | 0.30 | 90 | 15 |
| | 1.44 | 29 | 90 |
| | 1.66 | 26 | 95 |
| Ratio Combination^{a} | 0.83 | 95 | 20 |
| | 1.33 | 90 | 57 |
| | 2.22 | 42 | 90 |
| | 2.88 | 26 | 95 |

^{a}Ratio Combination function = 0.142 * % Free/Total PSA + 0.213 * % hK11/Total PSA as it was formulated from the logistic regression analysis.

TABLE 7
Serum hk11 concentration classified by tumor grade,
stage of the disease and Gleasson score

| | Total | Mean^{a} | Standard Error^{a} | Median^{a} | pvalue^{b} |
|---|---|---|---|---|---|
| Grade | | | | | |
| G1 | 8 | 0.171 | 0.016 | 0.166 | 0.036 |
| G2 | 90 | 0.152 | 0.009 | 0.145 | |
| G3 | 48 | 0.120 | 0.008 | 0.104 | |
| Stage | | | | | |
| I | 13 | 0.176 | 0.021 | 0.174 | |
| II | 88 | 0.141 | 0.078 | 0.133 | 0.038 |
| III/IV | 41 | 0.120 | 0.011 | 0.110 | |
| Gleason score | | | | | |
| ≦6 | 55 | 0.151 | 0.117 | 0.146 | |
| >6 | 72 | 0.131 | 0.008 | 0.125 | 0.176 |

^{a}ug/L
^{b}Calculated by the Mann-Whitney U Test

### FULL CITATIONS FOR REFERENCES REFERRED TO IN THE SPECIFICATION

1. Oesterling J E: Prostate specific antigen: a critical assessment of the most useful tumor marker for adenocarcinoma of the prostate. J Urol 145: 907-923, 1991.
2. Barry M J: Clinical practice. Prostate-specific-antigen testing for early diagnosis of prostate cancer. N Engl J Med 344: 1373-1377, 2001
3. Oesterling J E, Jacobsen S J, Klee G G, et al: Free, complexed and total serum prostate specific antigen: the establishment of appropriate reference ranges for their concentrations and ratios. J Urol 154:1090-1095, 1995.
4. Stephan C, Jung K, Lein M, et al: Molecular forms of prostate-specific antigen and human kallikrein 2 as promising tools for early diagnosis of prostate cancer. Cancer Epidemiol Biomarkers Prev 9: 1133-1147, 2000.
5. Catalona W J, Partin A W, Slawin K M, et al: Use of the percentage of free prostate-specific antigen to enhance differentiation of prostate cancer from benign prostatic disease: a prospective multicenter clinical trial. JAMA 279: 1542-1547, 1998.
6. Yoshida S, Taniguchi M, Suemoto T, et al: cDNA cloning and expression of a novel serine protease, TLSP. Biochim Biophys Acta 1399: 225-228, 1998.
7. Mitsui S, Yamada T, Okui A, et al: A novel isoform of a kallikrein-like protease, TLSP/hippostasin, (PRSS20), is expressed in the human brain and prostate. Biochem Biophys Res Commun 272: 205-211, 2000.
8. Nakamura T, Mitsui S, Okui A, et al: Alternative splicing isoforms of hippostasin (PRSS20/KLK11) in prostate cancer cell lines. Prostate 49: 72-78, 2001.
9. Yousef G M, Scorilas A, and Diamandis E P: Genomic organization, mapping, tissue expression, and hormonal regulation of trypsin-like serine protease (TLSP PRSS20), a new member of the human kallikrein gene family. Genomics 63: 88-96, 2000.
10. Diamandis E P, Yousef G M, Clements J, et al: New nomenclature for the human tissue kallikrein gene family. Clin Chem 46: 1855-1858, 2000.
11. Diamandis E P, Okui A, Mitsui S, et al: Human Kallikrein 11: A new biomarker of prostate and ovarian carcinoma. Cancer Res 62: 295-300, 2002.
12. Catalona W J, Partin A W, Finlay J A, et al: Use of percentage of free prostate-specific antigen to identify men at high risk of prostate cancer when PSA levels are 2.51 to 4 ng/mL and digital rectal examination is not suspicious for prostate cancer: an alternative model. Urology 54: 220-224, 1999.
13. Jung K, Elgeti U, Lein M, et al: Ratio of free or complexed prostate-specific antigen (PSA) to total PSA: which ratio improves differentiation between benign prostatic hyperplasia and prostate cancer? Clin Chem 46: 55-62, 2000.
14. Oesterling J B, Jacobsen S I, and Cooner W H: The use of age-specific reference ranges for serum prostate specific antigen in men 60 years old or older. J Urol 153: 1160-1163, 1995.
15. Magklara A, Scorilas A, Catalona W J, et al: The combination of human glandular kallikrein and free prostate-specific antigen (PSA) enhances discrimination between prostate cancer and benign prostatic hyperplasia in patients with moderately increased total PSA. Clin Chem 45: 1960-1966, 1999.

1. A method for detecting prostate cancer in a subject comprising measuring kallikrein 11 and prostate specific antigen (PSA) in a sample from the subject.

2. A method as claimed in claim 1 for detecting prostate cancer in a subject comprising:
(a) determining the amount of kallikrein 11 in a sample from the subject;
(b) determining the amount of PSA in the sample;
(c) mathematically combining the results of step (a) and step (b); and
(d) relating the combination to the presence of prostate cancer.

3. A method as claimed in claim 2 wherein in step (a) kallikrein 11 is determined using antibodies that specifically bind to kallikrein or a part thereof.

4. A method as claimed in claim 2, wherein in step (b) the PSA is measured using antibodies that specifically bind to PSA or a part thereof.

5. A method as claimed in claim 2 wherein in step (a) kallikrein 11 is determined in the sample by
(a) incubating a sample from the subject with a first antibody that specifically binds kallikrein 11 which is directly or indirectly labeled with a detectable substance, and a second antibody that specifically binds kallikrein 11 which is immobilized; and
(b) detecting the detectable substance thereby determining kallikrein 11 in the sample.

6. A method as claimed in claim 2, wherein in step (b) total PSA is determined in the sample.

7. A method as claimed in claim 2 wherein the combination is a ratio of kallikrein 11 to total PSA, or the inverse thereof.

8. A method as claimed in claim 2 which further comprises the step of determining the % free PSA and relating the combination and % free PSA to the presence of prostate cancer.

9. A method as claimed in claim 2 wherein the combination is compared to a predetermined standard.

10. A method for distinguishing prostate cancer from benign prostatic hyperplasia (BPH) in a subject comprising determining the amount of kallikrein 11 contained in a sample from the subject, and relating the amount to the presence of prostate cancer or BPH in the subject.

11. A method as claimed in claim 10 wherein the kallikrein 11 is measured using antibodies that specifically bind to kallikrein 11 or a part thereof.

12. A method as claimed in claim 10 wherein the amount of kallikrein 11 in the sample is compared to an amount determined for a standard.

13. A method as claimed in claim 12 wherein the standard is an amount of kallikrein 11 associated with prostate cancer.

14. A method as claimed in claim 13 wherein an amount of kallikrein 11 in the sample greater than the standard is indicative of BPH.

15. A method as claimed in claim 12 wherein the standard is an amount of kallikrein 11 associated with BPH.

16. A method as claimed in claim 15 wherein an amount of kallikrein 11 in the sample lower than the standard is indicative of prostate cancer.

17. The method for distinguishing prostate cancer from benign prostatic hyperplasia (BPH) according to claim 10, comprising:
(a) determining the amount of kallikrein 11 contained in a sample from the subject;
(b) determining the amount of total PSA contained in the sample;
(c) mathematically combining the results of (a) and (b);
(d) relating the combination to the presence of BPH or prostate cancer.

18. A method as claimed in claim 17 wherein the combination is a ratio of kallikrein 11 to total PSA, or the inverse thereof.

19. A method as claimed in claim 17 wherein the kallikrein 11 is measured using antibodies specifically reactive with kallikrein 11 or a part thereof.

20. A method as claimed in claim 17 wherein the PSA is measured using antibodies specifically reactive with PSA or a part thereof.

21. A method as claimed claim 17 wherein the combination is compared to a combination for a standard.

22. A method as claimed in claim 21 wherein the standard is a combination associated with prostate cancer.

23. A method as claimed in claim 22 wherein a combination in the sample is greater than the standard is indicative of BPH.

24. A method as claimed in claim 21 wherein the standard is a combination associated with BPH.

25. A method as claimed in claim 24 wherein a combination in the sample lower than the standard is indicative of prostate cancer.

26. A method of claim 17 further comprising determining the percentage of free PSA and correlating the percentage free PSA and the combination to the presence of prostate cancer or BPH in the subject.

27. A method for determining the presence of BPH or prostate cancer in a subject comprising:
(a) providing a first binding agent that specifically binds to kallikrein 11;
(b) providing a second binding agent that specifically binds to PSA;
(c) contacting the first agent and second agent with the sample under a condition that allows the formation of a first complex comprising the first agent and the kallikrein 11, and a second complex comprising the second agent and the PSA;
(d) determining the presence or amount of the first and second complexes;
(e) mathematically combining the amount of the first and second complexes; and
(f) relating the combination to the presence of BPH or prostate cancer.

28. A method as claimed in claim 27 wherein the combination is a ratio of the first complex to the second complex, or the inverse thereof.

29. A method of claim 27 wherein the binding agents are antibodies.

30. A method of claim 27 further comprising determining the percentage of free PSA and correlating the percentage free PSA and the combination to the presence of prostate cancer or BPH in the subject.

31. A method of claim 6 wherein the subject has total PSA between about 4-10 ng/ml.

32. A method of claim 6 wherein the subject has total PSA less than 4 ng/ml.

33. A method of claim 2 wherein the sample is a mammalian tissue sample.

34. A method of claim 2 wherein the sample is a sample of human physiological fluid.

35. A method of claim 2 wherein the sample is serum, seminal plasma, urine, or plasma.

36. A method of improving the accuracy of a diagnosis of prostate cancer comprising the steps of: a) performing a method of claim 2; and b) performing at least one of a test for free PSA and a digital rectal examination.

37. A method of claim 1 for detecting prostate cancer in a subject by determining the ratio between the measured kallikrein 11:total PSA in a serum sample of the subject.

38. A method for distinguishing prostate cancer from BPH in accordance with claim 17 wherein the sample is a serum sample and wherein step (c) comprises determining the ratio between kallikrein 11:total PSA in a serum sample of the subject.

39. (canceled)

40. A kit for determining the presence of BPH or prostate cancer in a subject, comprising:
(a) a known amount of a first binding agent that specifically binds to kallikrein 11; and
(b) a known amount of a second binding agent that specifically binds to PSA;
wherein the first and second binding agents comprise a detectable substance, or bind directly or indirectly to a detectable label.

41-49. (canceled)

50. A method of claim 10 wherein the subject has total PSA between about 4-10 ng/ml.

51. A method of claim 10 wherein the subject has total PSA less than 4 ng/ml.

52. A method of claim 10 wherein the sample is a mammalian tissue sample.

53. A method of claim 10 wherein the sample is a sample of human physiological fluid.

54. A method of claim 10 wherein the sample is serum, seminal plasma, urine, or plasma.


SEQUENCE LISTING (3 sequences)

SEQ ID NO: 1 - protein, 282 aa, homo sapiens
  ref <308>: DATABASE ACCESSION NUMBER: Genbank/AB012917
  ref <309>: DATABASE ENTRY DATE: 1998-04-10
  ref <313>: RELEVANT RESIDUES: (1)..(282)
MQRLRWLRDWKSSGRGLTAAKEPGARSSPLQAMRILQLILLALATGLVGGETRIIKGFECKPHSQPWQAALFEKTRLLCGATLIAPRWLLTAAHCLKPRYIVHLGQHNLQKEEGCEQTRTATESFPHPGFNNSLPNKDHRNDIMLVKMASPVSITWAVRPLTLSSRCVTAGTSCLISGWGSTSSPQLRLPHTLRCANITIIEHQKCENAYPGNITDTMVCASVQEGGKDSCQGDSGGPLVCNQSLQGIISWGQDPCAITRKPGVYTKVCKYVDWIQETMKNN

SEQ ID NO: 2 - DNA, 9120 bp, homo sapiens
  ref <308>: DATABASE ACCESSION NUMBER: Genbank/AF164623.
  ref <309>: DATABASE ENTRY DATE: 1999-07-01
  ref <313>: RELEVANT RESIDUES: (1)..(9120)
tgataatagtgttctctctcctcattggtcagggccccagccattgtccttgagagaatgctcgactctttatgttgtcttgacagcctcccctgagattggtcattaatgactgtgctctctctcctcattggtcagggccccagccattgtccttgagagaacctctgtcctttatggagttccacccttcttccctgggattggcccctagagacagtggttcttctcttttggttagccattgccattgtcctccgggaaagtgattatactcttttgtctaatgaccagacttggagccctccccaaggcccaggactgggttgaagggttggggaggaaaacagaaataagatgtctcccttgttcagacagtacttctcttcccttccagggtgattctgggggccccctggtgtgtgggggagtccttcaaggtctggtgtcctgggggtctgtggggccctgtggacaagatggcatccctggagtctacacctatatttgcaagtatgtggactggatccggatgatcatgaggaacaactgacctgtttcctccacctccacccccaccccttaacttgggtacccctctggccctcagagcaccaatatctcctccatcacttcccctagctccactcttgttggcctgggaacttcttggaactttaactcctgccagcccttctaagacccacgagcggggtgagagaagtgtgcaatagtctggaataaatataaatgaaggaggggccatgtctgtccatttgaagtcctcatgctggttgagactggaagaaggactcagcagtttccctatctcataggagtagaaacagagctcaaataaggccaggcacagtggctcacacctgtaatcccatcactttgggaagctgaggcaggtggatcacctgaggtcaggaactcgggaccagcctggtcaacatagtgaaaccccaactctactaaaaatgcaaaaattagccaggcatggtggcgcatgcctgtaatcccagctactcaggaggctgagacaggagaatagcatgaacccgtgaggcagaggctgcagcgagccgagattgaaccattacactccagcctgggcgacagagcgagactccatctcaaaaacaaacaaacaaaaaacccagtgctcaaataggatgagggtcttccctgagtagttactcagaaatggagtagaaaaagttacttttaataatataggccgggtgcagtggcccacgcctgtaatcccagcactttgggaggccgaggtgggaggatggcttgagctcagatttcgagatcagcctggcaacacagtgaaatcttgtcactacaaaaacacaaaaaattagctgggtgtggtggtgcgtgcctgtagtcccagctacttgggaagctgaggtgggaggatcacccgagccggggaggtggaggctgcaaagagccgagatcatgccactgcactccagcctgggcaataaagtgagaccttgtctcaaaaacaaaaacccagcaatataaataagacacatgtttcttcatctggcataatagaaatagtgcccagagcttataagcttttcaagagtccacaaaagacccgaaaaagaaaaagaaaattgttagctccaaaataccagatgaaagctgcaaagtcaacatttatgaccatttaatccaatgtccataaaacgtagcattctttccactagccaactgcagtttactttcttgtaatgaagcatacattgtatctttaatgtgggacgtggctttgttctaataagacgaagggtggagtgcaggcttggaaagcaggagagctcagcctacgtctttaatcctcctgcccaccccttggattctgtctccactgggactcaagaggtgaggagagaccatctccccaaatgcactgaagggaaactggaggagggagggagtgaggggtgatcataccagcggaggcacatttgctgagcccccccgcagtctgctctttccaagtggaccctcctggaagcctgatcccaacctcccctgcaagcaggtctgtcacccccatctctcagatgaagaaactgagccttgcaggggtggagtcccttgtccccacgtcataagggtagtcatagtagtaggaagaggaagcacctaggtttgaggccagggctggctgctgtcagaacctaggccctcccctgccttgctccacacctggtcaggggagagaggggaggaaagccaagggaagggacctaactgaaaacaaacaagctgggagaagcaggaatctgcgctcgggttccgcagatgcagaggttgaggtggctgcgggactggaagtcatcgggcagaggtctcacagcagccagtaagtgaacagctggactcgggctgcctgggcggcagggagaagcgggcaggggaagggtcagcagaggagcgaggccccagaggagccctggggtggagcacagccaagggctctgttccctttcctggactcggcttccacaggccctgacctgcctcccccaccctccggtcctgcccctgtgcctggcagcagccccacctgtgtgacatcccagcacaccccccctctccttgcaaaggagaagggagcggcctaggggaggccaggggcccacctgggctggggctgtggagagggagtggctgggacgggaggaaaaagagagacggagattagatggaagaagagggatttcaagacaaattgccagagatgcagtcagagagactgactgagagacacaaagatagaaggaattagagaaagggccacacagagccagacagagagagaagagtggagatggagacagggacgaggacagagaaaggcagacagacacatagggacagaaagagaaaaatcacacaaagtcagaattactgaatgacagggaatgacacatagaacgagacacagattcagagactcagggcagggaaaggaaggctgcagacagacagacagacagagggaggctgagacacagggagaagaggggcttggagaggtggcacaggcaggcagccagtgcctcagaggcctccggggagggccctcacacacaccccgccccggggcattaaggcagggcttggaggccagtcatcctgggcccgcccagggccgcccccctgccagcccgcctgcctggtgcctggcacctggcgctccaacccagcctacctgctgtagctgccgccactgccgtctccgccgccactgggcccccagagccccagccccagagcctgtgagtccaggaggaaagggaagctgcccctccccgtccaggtgtcagccctccccaaggacacctgtcccactcgggcacccatttctccctctgctctgtccttctctgcttgggtgggggttcctggcctctctctacacctctcacctccgatggctgtccgcagcctcagttacctctaatctccatggcttcagctgctgagctggccctctgctcccacccccgctggccagggcagcggagggcactggccctcccctcgaccagccccgcccagctttgcttggctgtccttcaaaagggcaggggtttggcggacagggcttcagcaagccgggtgatgggggtcccagacattgtctggggctgagccccctactcccctccagcagacctcaaaggctccatatcgctctgctgcgaagacaatgaaaaaggggtggctacggaacggtgtctggttccccttgtccttccaccccaagctgctggggcctggccagctctcaaggcaagaaggaaaacatcctctgacatgtgccggggaggtcccatggctgacttgaacagggccgaaccatggcttgacagctcaaagcccctcccaacgacttccacatggttcttggtatctcggaagcttctagctgtgaccaggccctctccaaggccaccctagacacctaagatatattttaagtgtttggagatctgagtgctgtgagaaacaggggatttccccaaccttgtttctcccaagtggggagcgggagcaggtgagggagagaggagagggcatgagccagcccccccctcccgatttccccgtaaagtgatgcggccccatgtccctccttgttcccagaggaacctggggcccgctcctcccccctccaggccatgaggattctgcagttaatcctgcttgctctggcaacaggtacgcaggggatgggggcagggcaggatcctccctcttgaatctctgggatcccctaaccctctgtgtctggacagtgacagggctgattccaaattacagaacaacccataaggcacctgaactggagcagtggtcatgagggcctggatgcccttctagataatccctttaaatgccaaaggaggagaggtcaagggggtcgtaaagggtcccgtggaggggctgaggaagctggagttgggggagcagtcactcaaagcgcccaggacaggggctactgaccaaccagtatggaagtatttccttttttttttttcccagagacaaagtcttgctctattgtccaggctggagtgccgtggtgccaacacggctcactgcagtcttgacttcccgggcttaagtgatccttaagccatctcagcttccccggtagctgggaccacaggcacctgccaccaagccaggctaattgtttaattgtttgtagagatgggggaggaggtctcactatgtttgcctaggctgatctagaactcctgggctcaagtaatcctcccaccttagcctctcaaagtgctgggattacaggcatgagccactgcatttgaccttatggaagtattttcatcctttaatacccgaccccagcatccagggcaacccagagggacaccagaccagggcccagaccacccactctctttctctcctccccacccccatttctgggagtcctcctggtctaccacctctccttcctgagccccttcttttgctctcaccccctccagggcttgtagggggagagaccaggatcatcaaggggttcgagtgcaagcctcactcccagccctggcaggcagccctgttcgagaagacgcggctactctgtggggcgacgctcatcgcccccagatggctcctgacagcagcccactgcctcaagccgtgggtgcgggggctggggcggtgccggggtggggggctgggaatggggagatggatggagagaagctcagggataggggtgctggtaaggggattagagatggggatgggtagtgtcagcaaggttgatgggctcgagttggtattgaaggtggggggatgaatggggttgggatggggctatggctgggaagggggcttcggtgggagacgtggaagaggttggaagcagagcgatgtttcttcatcctcaaaggtgtcactcacctctcccacccatgtctcccccgacctttcctcctccaactactgtctctcccacctcagccgctacatagttcacctggggcagcacaacctccagaaggaggagggctgtgagcagacccggacagccactgagtccttcccccaccccggcttcaacaacagcctccccaacaaagaccaccgcaatgacatcatgctggtgaagatggcatcgccagtctccatcacctgggctgtgcgacccctcaccctctcctcacgctgtgtcactgctggcaccagctgcctcatttccggctggggcagcacgtccagcccccagtgtaggagcaccagaggggaacctggcagggggtggtgaggagggagtggtcaggattgtggaagggttcagggcatcagagatgcggttcacagtgacgatgtgggataagttgagaggatgtgtggaaaacgtcaggataggggggtggggacaaaagttggggccttggagtcagacggacgggatatgcaatcatacatccataacctcctggttgtaagaccttaggcaagcagcttcacctctctgaatcttgattttcttctctataaaatgagaatgattatacccacctgtcaggattggattagagataatgtatatcaagcaactgacataaatcatttattggatagcaggctgggcaccgtggctcacgcctgtaatcccagcactttgggaggccgaggtgggaagatcacctgaggtcaggactttgataccagcctggccaacgtggtgaaatcccatctctactaaaaatgtgaaaattagttgggcgtggttgtgtgcgcctgtaatcccagctactcgggaggttgaggcaggagaatcgcttaaacttgggagacggaggttgcagtgagccaagatcacgccactgcactccagcctgggcaacagagcaagactctgtctcgaaaaaaaaaaaaaaaaagctggatagcattgctgttgctattgttacaagaagagaggtgagttggctgcgtctaaggacagggattcccccaggggcgggatcacagcaagcactgcattaggggaggtggcagggggctcattcccacagcccctcacgctgtttccacagtacgcctgcctcacaccttgcgatgcgccaacatcaccatcattgagcaccagaagtgtgagaacgcctaccccggcaacatcacagacaccatggtgtgtgccagcgtgcaggaagggggcaaggactcctgccaggtcagtgtggtctccaaccacagccccatccccatccccagcttcaatgacatctttaccgacatccacaatttcatccccaacctcaacccgccgacccctgcaactcccaatccatctcttcccctgttcccgtttctgacctcagcacaaacttcagctccatccccgtttccacaccatttccagctccaaccatccccaaactcgtttttgagcctaaccccatcctttatcccacccataatcccagctttatcgctaaacctatcacctttcccagtgcctacccatcctgtctcggccccactcctaagcaccgtccccacctcctccctggctaacaccatgctcaacgctttctctgaccgacattctctctccccgtgcccagggtgactccgggggccctctggtctgtaaccagtctcttcaaggcattatctcctggggccaggatccgtgtgcgatcacccgaaagcctggtgtctacacgaaagtctgcaaatatgtggactggatccaggagacgatgaagaacaattagactggacccacccaccacagcccatcaccctccatttccacttggtgtttggttcctgttcactctgttaataagaaaccctaagccaagaccctctacgaacattctttgggcctcctggactacaggagatgctgtcacttaataatcaacctggggttcgaaatcagtgagacctggattcaaattctgccttgaaatattgtgactctgggaatgacaacacctggtttgttctctgttgtatccccagccccaaagacagctcctggccatatatcaaggtttcaataaatatttgctaaatgagtgaatctactgagtgcttactatgtgctagaccctgatccaatggcttttattttattttattttttgacagagtctcgctctgtcacccaggctggagtacagtggtgctatctctgctcactgcaacctccacctcctgggttcaagcaattctcctgcctcagcctcctgaatagctgggattacaggtgcctaccaccacatccggctaatttttgtattttttagtagagatggggcttcaccatgttggccaggctggtctcgaactcctgacctcagatgatctgccctccttggcctcccaaactcctgggattacagacgtgagccaccgcgcccgcccggctttcatttattaattaaaagaaattaaattaattaatctatttaggagacagtcttgctctgttgcccaggctggagtgcagtaacaatcacagctcacggcaatctcaatttcctggggtcaagtgattgtcctccctcagcctccagagtagctgggactacaggcacatgccacgaagcccagctaatttttgtatttttcgtagagacagaggtctcagtatgttgccccggctagtctcaaactcctgggctcaagcagtctgtcctcctcagcctccaaaagtggtgagattacaggcatgagtcgctgtgcctggcctccaagcactttcaaatgtatcaacttaatcctcacaaaaccctgtgaggtcggtactgttttcatacctattttatagttgaagaaacagacacagagaagcaaagtcacttgctcacagtcacgtggctaggagagcaaggatctgaagcaaggcgatctcttaattaccaagtgatgttcctggagtaaggctctgtttgtttcctttcctgtaaaatgctgcatgcaaaagtataacacagtaagtaaagaagtcagttagcctgcacatactaagacctaaccaaaggagcttattgtttttctccaacttccatgataggtaattagatagtggagacctctgctggccaatatggtagccactaaccgcagctggctcttccaattaaaattacataaagccagaaatgtaactcctctgtctcacttgttatatctccaaggctggatagccacatgtgactggtggtggctggattagctagtgcatataaaacatcactgcagaaagttcagctgagcagcactgagttagatggcctctgaagaggatgtcccacggagagaatccagaactcaggatctttttttttttttttctttgcgacagagtcttgctctgtcacccaggctggagtgcagtggcgtgatctcggctcactgcaacttctgcctcccaggttcaagcaattctcctgcctcagcctccctagtagctgggactacaggcctgtgccaacatccccagctaatttttgtgtctttttagtagagatggggtttcactatgttggccaggctggtctcgaactcctgacc

SEQ ID NO: 3 - DNA, 1186 bp, homo sapiens
aggaatctgcgctcgggttccgcagatgcagaggttgaggtggctgcgggactggaagtcatcgggcagaggtctcacagcagccaaggaacctggggcccgctcctcccccctccaggccatgaggattctgcagttaatcctgcttgctctggcaacagggcttgtagggggagagaccaggatcatcaaggggttcgagtgcaagcctcactcccagccctggcaggcagccctgttcgagaagacgcggctactctgtggggcgacgctcatcgcccccagatggctcctgacagcagcccactgcctcaagccccgctacatagttcacctggggcagcacaacctccagaaggaggagggctgtgagcagacccggacagccactgagtccttcccccaccccggcttcaacaacagcctccccaacaaagaccaccgcaatgacatcatgctggtgaagatggcatcgccagtctccatcacctgggctgtgcgacccctcaccctctcctcacgctgtgtcactgctggcaccagctgcctcatttccggctggggcagcacgtccagcccccagttacgcctgcctcacaccttgcgatgcgccaacatcaccatcattgagcaccagaagtgtgagaacgcctaccccggcaacatcacagacaccatggtgtgtgccagcgtgcaggaagggggcaaggactcctgccagggtgactccgggggccctctggtctgtaaccagtctcttcaaggcattatctcctggggccaggatccgtgtgcgatcacccgaaagcctggtgtctacacgaaagtctgcaaatatgtggactggatccaggagacgatgaagaacaattagactggacccacccaccacagcccatcaccctccatttccacttggtgtttggttcctgttcactctgttaataagaaaccctaagccaagaccctctgcgaacattctttgggcctcctggactacaggagatgctgtcacttaataatcaacctggggttcgaaatcagtgagacctggattcaaattctgccttgaaatattgtgactctgggaatgacaacacctggtttgttctctgttgtatccccagccccaaagacagctcctggccatatatcaaggtttcaataaatatttgctaaatgagtg